(19) United States
(12) Patent Application Publication
(10) Pub. No.: US 2025/0222185 A1
(43) Pub. Date: Jul. 10, 2025

(54) DEVICES FOR BLOOD CAPTURE AND REINTRODUCTION DURING ASPIRATION PROCEDURE

(71) Applicant: Imperative Care, Inc., Campbell, CA (US)

(72) Inventors: Michael Buck, Menlo Park, CA (US); Julia Fox, San Carlos, CA (US); James Jacobs, Danville, CA (US)

(21) Appl. No.: 18/947,732

(22) Filed: Nov. 14, 2024

Related U.S. Application Data
(63) Continuation-in-part of application No. 18/407,218, filed on Jan. 8, 2024, now Pat. No. 12,171,917.
(63) Continuation-in-part of application No. 18/407,188, filed on Jan. 8, 2024.
(63) Continuation-in-part of application No. 18/407,214, filed on Jan. 8, 2024.

(51) Int. Cl. A61M 1/34; A61M 1/00; A61M 39/24

(52) U.S. Cl. CPC A61M 1/34; A61M 1/63; A61M 39/24; A61M 2202/0007; A61M 2202/0014; A61M 2202/0021; A61M 2202/0028; A61M 2202/0042; A61M 2202/005; A61M 2202/0413; A61M 2202/06; A61M 2205/10; A61M 2205/7545

(57) ABSTRACT

A blood reintroduction system can include a canister having a chamber configured to collect blood. A system may include an inlet configured to fluidically connect the chamber to a first tubing in fluid communication with an aspiration catheter. A system may include a first filter within the chamber, wherein the first filter is configured to separate blood from thrombus. A system may include a second filter downstream of the first filter, wherein the second filter is configured to separate blood from thrombus. A system may include a second tubing connected to an outlet of the chamber, wherein the second tubing is configured to reintroduce filtered blood from the chamber to a patient's vasculature.

### CROSS-REFERENCE TO RELATED APPLICATIONS

This application is a continuation-in-part of U.S. patent application Ser. No. 18/407,188, filed Jan. 8, 2024; U.S. patent application Ser. No. 18/407,214, filed Jan. 8, 2024; and U.S. patent application Ser. No. 18/407,218, filed Jan. 8, 2024, the entirety of which are hereby incorporated by reference herein.

### BACKGROUND

Thrombotic restrictions and occlusions within a patient's blood vessels are a significant medical problem and often require intervention to remove these restrictions and blockages to restore health to patients. While applicable to a wide range of vascular applications in both the arterial and venous systems, including a variety of small vessels, the following background illuminates the problems primarily through the example of patients suffering with Pulmonary Embolisms.

Venous thromboembolic disease (VTE) is a worldwide crisis. There are over 10 million cases of deep vein thrombosis (DVT) and pulmonary embolism (PE) diagnosed globally per year, with 1 million cases occurring in the United States and over 700,000 in France, Italy, Germany, Spain, Sweden, and the United Kingdom combined each year. There are approximately 60,000 to 100,000 deaths from PE in the United States each year. DVT and PE are part of the same continuum of disease, with over 95% of emboli originating in the lower extremities. When PE occurs, the severity depends on the embolic burden and its effect on the right ventricle as well as underlying cardiopulmonary comorbidities. Death can result from the acute increase in pulmonary artery (PA) pressure with increased right ventricular (RV) afterload and dysfunction.

Patients with high-risk pulmonary embolism (PE) were treated primarily with thrombolytic therapy delivered systemically or more locally through Catheter Directed Thrombolytics. These approaches result in multiple catheterization lab visits, lengthy hospital stays and often lead to bleeding complications. Newer approaches to PE treatment include single session thrombectomy treatments without the use of thrombolytics. These thrombectomy treatments include delivering a catheter into the PA to remove the thrombus through aspiration, and secondary tools may also macerate or disrupt the thrombus prior to aspiration. While thrombectomy results in fewer bleeding complications and reduced hospital stays compared to thrombolytics, there is much to be improved upon given the challenges of the procedure itself, including the ability to capture a broad spectrum of thrombus types and reduce the total volume of blood loss during the procedure.

The thrombectomy catheter is introduced through an introducer puncture in a large diameter vein. A flexible guide wire is passed through the introducer into the vein and the introducer is removed. The flexible guidewire provides a rail for a flexible guide catheter to be advanced through the right atrium into the right ventricle and into the pulmonary artery. The flexible guidewire is removed and replaced with a stiff guidewire. The large diameter thrombectomy catheter with support dilator is then advanced over the stiff guidewire to the pulmonary artery and the dilator is removed. If the large diameter thrombectomy catheter is not successful in accessing or aspirating thrombus in a more distal portion of the vessel, a smaller diameter catheter may be inserted through the large diameter catheter.

In addition, peripheral arterial occlusive (PAO) disease occurs in more than 4% of individuals over age 40 and markedly increases in incidence after the age of 70. Acute PAO is usually due to thrombosis of the peripheral vasculature and is associated with a significant risk of limb loss. In order to preserve the limb, therapy for acute PAO centers on the rapid restoration of arterial patency and blood flow such as through mechanical thrombectomy in procedures similar to those described above.

Clot aspiration using certain commercial vacuum-assisted thrombectomy systems may sometimes need to be terminated due to the risk of excessive blood loss by the patient, especially when using large aspiration catheters. During aspiration thrombectomy, when the catheter tip falls out of contact with the thrombus or other occlusive material, the tip is exposed to healthy blood and full flow of blood through the catheter ensues. Under such conditions, the total volume of blood loss is excessive, and in some cases, may result in premature termination of the procedure. For example, during a procedure when the catheter enters healthy blood and full aspiration flow ensues, the blood loss rate can be on the order of 20-30 cc per second with an 24 French size catheter. In order to minimize blood loss, the catheter should not run in unrestricted mode for more than approximately 10 to 15 seconds. The aggregate blood loss may reach an unacceptable level before sufficient clot is removed.

### SUMMARY

There is provided in accordance with one aspect of the present disclosure a blood reintroduction system. The blood reintroduction system can include a sterile canister configured to collect blood, an inlet configured to be fluidly connected to a first tubing in fluid communication with an aspiration system configured to apply aspiration to a vasculature of a patient, a first outlet configured to be fluidly connected to a second tubing in fluid communication with an aspiration pump, and/or any vacuum source such as a syringe, and a second outlet configured to interact with a blood reintroduction device. The blood reintroduction device can be configured to withdraw the blood collected inside the sterile canister. The blood reintroduction device can include a filter positioned inside a flow path extending through the second outlet.

In some aspects, the filter can be positioned anywhere between the sterile canister and the vasculature of the patient.

In some aspects the filter is positioned inside the sterile canister.

In some aspects, the filter is positioned outside the sterile canister.

In some aspects, the filter is in fluid communication with the syringe.

In some aspects, the filter is in fluid communication with patient tubing and/or a patient port.

The inlet may be configured to direct an incoming stream of blood along an inside surface of the canister. In one implementation, the canister may have a curved wall such as in a cylindrical canister. The inlet may be configured to direct an incoming stream of blood along a circumferential path along the inside surface of the canister.

In some aspects, the filter is positioned between a chamber defined by the sterile canister and an opening of the second outlet. The filter can be configured to filter the blood before the blood is withdrawn from the sterile canister via the second outlet.

In some aspects, the inlet can be positioned on an upper portion of the sterile canister.

In some aspects, the first outlet can be positioned on an upper portion of the canister.

In some aspects, the canister further includes a base defining a blood collection cavity.

In some aspects, the filter is at least partially disposed inside the cavity. The second outlet can be positioned adjacent to the cavity.

In some aspects, the blood reintroduction device includes a syringe.

In some aspects, the blood reintroduction device includes a venous line in fluid communication with vasculature of the patient. A pump fluidly connected to the venous line can be configured to advance the blood withdrawn from the sterile canister into the vasculature of the patient.

In some aspects, the aspiration system includes a thrombectomy catheter and an aspiration catheter configured to be advanced through the thrombectomy catheter.

In some aspects, aspiration from the aspiration pump or any vacuum source, such as a syringe, is configured to draw blood from the first tubing into the sterile canister.

In some aspects, a longitudinal axis of the second outlet is positioned at an acute angle relative to a base of the sterile canister.

In some aspects, a longitudinal axis of the second outlet is positioned at a right angle relative to a base of the sterile canister.

In some aspects, the first and second tubing are configured to permit the sterile canister to reside within a sterile field.

In accordance with another aspect of the present disclosure, there is provided a method of capturing blood for reintroduction into a vasculature of a patient during an aspiration procedure. The method can include providing a canister to be placed in fluid communication with the vasculature of a patient. The canister can include an inlet, a first outlet, and a second outlet. The method can also include applying aspiration to the canister via the first outlet, and aspirating blood from the patient and into the canister via a first tubing connected to the inlet. The method can include filtering the blood collected inside the canister using a filter positioned in fluid communication between an opening of the second outlet and at least a portion of an internal space of the canister. The method can include withdrawing filtered blood from the canister via the second outlet.

In some aspects, the method can include introducing the filtered blood withdrawn from the canister into the vasculature of a patient.

In some aspects, applying aspiration to the canister via the first outlet includes connecting the first outlet with an aspiration pump via a second tubing.

In some aspects, aspirating blood from the patient and into the canister via the first tubing connected to the inlet includes inserting a thrombectomy catheter into the vasculature of the patient and advancing an aspiration catheter inside the thrombectomy catheter.

In some aspects, filtering the blood collected inside the canister using the filter positioned between the opening of the second outlet and at least the portion of the internal space of the canister includes flowing the blood through the filter to cause filtering of solid material.

In some aspects, the solid material comprises a blood clot and/or a thrombus.

In some aspects, withdrawing filtered blood from the canister via the second outlet includes coupling a syringe to the second outlet and withdrawing blood into the syringe.

In some aspects, withdrawing filtered blood from the canister via the second outlet includes connecting a first end of a venous line into the vasculature of the patient and a second end of the venous line to the second outlet. The method can also include pumping the filtered blood through the venous line using a pump fluidly connected to the venous line.

In some aspects, filtering the blood collected inside the canister using the filter positioned between the opening of the second outlet and at least the portion of the internal space of the canister includes collecting solid matter inside the filter.

In some aspects, withdrawing filtered blood from the canister via the second outlet can be done without interrupting application of aspiration to the canister via the first outlet.

In some aspects, the method can include prefiltering the blood prior to aspirating the blood into the canister using a second filter positioned along the first tubing.

In some aspects, filtering the blood collected inside the canister using the filter includes filtering solid materials of a first size, prefiltering the blood prior to aspirating the blood into the canister using the second filter includes filtering solid materials of a second size. In some aspects, the second size can be greater than the first size. In some aspects, the solid materials can include a thrombus.

In some aspects, aspirating blood from the patient and into the canister can include positioning the inlet to direct a flow of blood to an interior wall of the canister.

In some aspects, aspirating blood from the patient and into the canister includes collecting the blood within a base of the canister, the base comprising a first portion and a second portion, the first portion comprising an inclined surface and the second portion defining a dip.

In some aspects, filtering the blood collected inside the canister using the filter includes positioning at least a portion of a distal end of the filter within the dip so that the distal end of the filter is in contact with the blood collected in the dip.

In accordance with another aspect of the present disclosure, there is provided a blood reintroduction system. The blood reintroductions system can include a housing having a chamber configured to collect blood, an inlet configured to fluidly connect the chamber to a first tubing in fluid communication with an aspiration catheter, a first outlet configured to fluidly connect the chamber to a second tubing in fluid communication with an aspiration pump, and a second outlet configured to interact with a blood reintroduction device.

In some aspects, the blood reintroduction system is configured to reside within a sterile field.

In some aspects, the blood reintroduction system can include a filter positioned between an interior portion of the housing and an opening of the second outlet.

In some aspects, the blood reintroduction device is configured to withdraw blood collected inside the housing.

In some aspects, the blood reintroduction device includes a syringe.

In some aspects, the second outlet includes a luer fitting.

In some aspects, the aspiration catheter is configured to apply aspiration to a vasculature of a patient.

In some aspects, the housing includes a base defining a floor. The floor can include a first portion and a second portion. The first portion can include an inclined surface and the second portion can define a dip. The inclined surface of the first portion can facilitate flow of the blood collected inside the housing towards the dip defined by the second portion.

In some aspects, the blood reintroduction system can further include a filter. The filter can include a proximal end and a distal end. The distal end of the filter can be positioned at least partially within the dip defined by the second portion of the floor.

In some aspects, the blood reintroduction system can further include a first filter and second filter. The first filter can have pores with a larger diameter than the second filter.

In some aspects, the blood reintroduction device can be configured to withdraw blood collected inside the canister while aspiration is applied by the aspiration pump.

In some aspects, the blood reintroductions system can include a filter positioned between the inlet and the aspiration catheter, the filter configured to capture thrombus as blood flows through the first tubing.

In some aspects, the blood reintroduction device includes a second tubing comprising a first end and a second end, the first end connected to the second outlet and the second end in fluid communication with a vasculature of a patient.

In some aspects, the second tubing can be in fluid communication with a pump, the pump configured to move the blood from the chamber to the vasculature of the patient via the tubing.

In some aspects, the blood reintroduction device includes a filter assembly having a filter housing, a filter positioned inside the filter housing and a cap configured to secure the filter housing to the housing of the blood reintroduction system.

In some aspects, the cap can be movable between at least an open position where the filter housing is not secured to the housing of the blood reintroduction system, and a closed position where the filter housing is secured to the housing of the blood reintroduction system.

In some aspects, at least a portion of the filter assembly can be positioned within the chamber and wherein at least a portion of the filter is in contact with the blood when blood is collected inside the chamber.

In accordance with another aspect of the present disclosure, there is provided a canister for use in a blood reintroduction system. The canister can include a housing, a base including a floor, the floor defining an inclined surface and a blood collection low point. The housing and the base can define a chamber configured to collect blood. The canister can include an inlet configured to be fluidly connected to a first tubing in fluid communication with a thrombectomy catheter. The inlet can be oriented to direct flow of blood along an interior side wall of the housing. The canister can include a first outlet configured to be fluidly connected to a second tubing in fluid communication with an aspiration pump, and a second outlet configured to interact with a blood reintroduction device. The blood reintroduction device can be configured to withdraw the blood collected inside the chamber. The canister can include a filter disposed between an opening of the second outlet and the chamber.

In some aspects, the inlet and the first outlet are positioned on an upper portion of the housing.

In some aspects, the inlet and the first outlet are positioned in an elevated position relative to the second outlet.

In some aspects, the filter includes a proximal end and a distal end. The distal end of the filter can be positioned adjacent the blood collection low point.

In some aspects, the filter and a bottom surface of the base define an acute angle.

In accordance with another aspect of the present disclosure, there is provided a filter assembly for use in a blood reintroduction system. The filter assembly can include a filter housing including a proximal end and a distal end, a cap removably secured to the proximal end of the filter housing, and a filter including a proximal end and a distal end. The filter can be positioned inside the filter housing. The filter assembly can include a luer fitting secured to the cap and configured to be in fluid communication with a blood reintroduction device. The filter housing can be configured to be at least partially disposed inside a canister.

In some aspects, a longitudinal axis of the filter housing can be positioned at an acute angle relative to a base of the canister.

In some aspects, the filter includes a filter having a pore size between about 20 and about 400 microns.

In accordance with another aspect of the present disclosure, there is provided a blood reintroduction system. The blood reintroduction system can include a canister having a chamber configured to collect blood; an inlet configured to fluidically connect the chamber to a first tubing in fluid communication with an aspiration catheter; a first filter within the chamber, wherein the first filter is configured to separate blood from thrombus; a second filter downstream of the first filter, wherein the second filter is configured to separate blood from thrombus; and a second tubing connected to an outlet of the chamber, wherein the second tubing is configured to reintroduce filtered blood from the chamber to a patient's vasculature.

The blood reintroduction system of any of the preceding paragraphs and/or any of the apparatuses, systems, or devices disclosed herein can include one or more of the following features. In some aspects, the techniques described herein relate to a blood reintroduction system, wherein a porosity of the first filter can be between about 50 microns and about 2000 microns. A porosity of the second filter can be between about 20 microns and about 120 microns. In some cases, the blood reintroduction system can be configured to reside within a sterile field. In some aspects, the second filter can be positioned outside the chamber and along the second tubing. The second filter can be located within the chamber below the first filter. In some cases, the blood reintroduction system can include a third filter positioned outside the chamber and along the second tubing. In some aspects, the blood reintroduction system can include a pump configured to decrease pressure within the chamber. The blood reintroduction system can include a syringe configured to decrease pressure within the chamber. In some cases, the second tubing can be fluidically connected to a floor of the canister. In some aspects, the floor is positioned on a bottom portion of the canister. The outlet can include a luer fitting.

In some cases, the blood reintroduction system can include an aspiration catheter configured to apply aspiration to the vasculature of the patient. In some aspects, the canister can include a floor. The floor can include at least one sloped portion; the at least one sloped portion can be configured to direct flow of the blood collected inside the canister towards the outlet. In some aspects, the blood reintroduction system can include a third tubing including a first end and a second end, the first end configured to be in fluid communication with the second tubing and the second end configured to be in fluid communication with the vasculature of the patient. The third tubing can be configured to be in fluid communication with an aspiration source, and the aspiration source is configured to move the blood from the chamber to the vasculature of the patient via the third tubing.

In some aspects, the blood reintroduction system can include a first one-way valve along the second tubing and a second one-way valve along the third tubing. The first one-way valve can be configured to prevent flow from the third tubing to the second tubing. In some cases, the second one-way valve can be configured to prevent flow from a first portion of the third tubing distal to the second one-way valve to a second portion of the third tubing proximal to the second one-way valve.

In another aspects, a method of reintroducing blood to a patient's vasculature can include providing a canister to be placed in fluid communication with an aspiration catheter; applying aspiration via the aspiration catheter to remove blood and thrombus material from the patient's vasculature; drawing the blood and thrombus material removed by the aspiration catheter into a chamber within the canister; passing the blood through a first filter and a second filter, wherein the first and second filters are configured to separate blood from thrombus; collecting the blood in a portion of the chamber positioned above a base of the canister; and directing the blood via a sloped floor to an outlet fluidically connected to a second tubing, wherein the second tubing is configured to reintroduce filtered blood to the patient's vasculature.

The method of reintroducing blood to a patient's vasculature can include one or more of the following features and/or steps. In some aspects, drawing the blood through the first filter includes drawing the blood through a filter with a porosity of between about 50 microns and about 2000 microns. Drawing the blood through the second filter can include drawing the blood through a filter with a porosity of between about 20 microns and about 120 microns. In some cases, the techniques described herein relate to a method, the canister resides within a sterile field while drawing the blood and thrombus material removed by the aspiration catheter into the chamber within the canister. In some aspects, the second filter is located within the chamber below the first filter. The method can include directing the blood passing through the second filter down at least one sloped portion of a floor of the canister, wherein the at least one sloped portion is configured to direct flow of the blood collected inside the canister towards the outlet of the canister.

In some cases, the method includes drawing filtered blood through a third tubing including a first end and a second end, the first end in fluid communication with the second tubing and the second end in fluid communication with the vasculature of the patient. The method can include drawing filtered blood through the third tubing with an aspiration source, wherein the aspiration source is configured to move the blood from the chamber to the vasculature of the patient via the third tubing. In some cases, the method can include drawing the filtered blood through a first one-way valve along the second tubing and a second one-way valve along the third tubing. The first one-way valve can be configured to prevent flow from the third tubing to the second tubing.

In another aspects, a method of reintroducing blood to vasculature, can include providing a canister to be placed in fluid communication with an aspiration catheter; applying aspiration via the aspiration catheter to remove blood and thrombus material from a patient's vasculature; collecting the blood and thrombus material removed by the catheter inside a chamber of the canister; drawing the blood through a first filter, wherein the first filter is configured to separate blood from thrombus; drawing the blood through a second filter, wherein the second filter is configured to separate blood from thrombus; collecting the blood in a second portion of the chamber positioned above a base of the canister; directing the blood via a sloped floor to an outlet fluidically connected to a second tubing; and reintroducing the filtered blood to a patient's vasculature.

The method of reintroducing blood to a patient's vasculature can include one or more of the following features and/or steps. In some aspects, drawing the blood through the first filter includes drawing the blood through a filter with a porosity of between about 50 microns and about 2000 microns. Drawing the blood through the second filter includes drawing the blood through a filter with a porosity of between about 20 microns and about 120 microns. In some cases, the canister can reside within a sterile field while collecting the blood and thrombus material removed by the aspiration catheter inside a chamber within the canister. In some aspects, the second filter can be positioned outside the chamber and along the second tubing.

In another aspects, a method of reintroducing blood to vasculature can include providing a canister to be placed in fluid communication with an aspiration catheter; applying aspiration via the aspiration catheter to remove blood and thrombus material from a patient's vasculature; collecting the blood and thrombus material removed by the catheter inside a chamber of the canister; drawing the blood through a first filter, wherein the first filter is configured to separate blood from thrombus; collecting the blood in a second portion of the chamber positioned above a base of the canister; directing the blood via a sloped floor to an outlet fluidically connected to a second tubing; subsequently drawing the blood through a second filter, wherein the second filter is configured to separate blood from thrombus; and reintroducing the filtered blood to a patient's vasculature.

The method of reintroducing blood to a patient's vasculature can include one or more of the following features and/or steps. In some aspects, drawing the blood through the first filter includes drawing the blood through a filter with a porosity of between about 50 microns and about 2000 microns. Drawing the blood through the second filter includes drawing the blood through a filter with a porosity of between about 20 microns and about 120 microns. In some cases, the canister can reside within a sterile field while collecting the blood and thrombus material removed by the aspiration catheter inside a chamber within the canister.

### BRIEF DESCRIPTION OF THE DRAWINGS

FIG. 1 is a schematic view of an alternative aspiration system having a first thrombectomy catheter and a second thrombectomy catheter extending therethrough.

FIG. 2A is a schematic view of the proximal handle for the first thrombectomy catheter of FIG. 1.

FIGS. 2B-2E illustrate interface details between a filter assembly and a proximal handle.

FIG. 3A is a schematic view of the proximal handle for the second thrombectomy catheter of FIG. 1.

FIG. 3B is a simplified flow diagram of the dual vacuum chamber aspiration system.

FIG. 3C is a qualitative fluid flow rate diagram at the catheter tip, following opening of the momentary vacuum control valve.

FIG. 4 is a schematic flow diagram for a three-way valve.

FIGS. 5A-5C illustrate three flow configurations for a three-way valve.

FIGS. 6A-6C illustrate operation of a hemostasis valve.

FIG. 6D illustrates an alternative filament configuration of the hemostasis valve.

FIGS. 7A-7B are schematic layouts of the components of a proximal handle of an aspiration catheter.

FIGS. 8A and 8B are different implementations of thrombus engagement tools.

FIG. 9A is a side elevational view of one thrombus engagement tool tip.

FIG. 9B is a longitudinal cross-section through the tip of FIG. 9A.

FIG. 10A is a side elevational view of an alternative thrombus engagement tip.

FIG. 10B is a longitudinal cross-section through the tip of FIG. 10A.

FIGS. 11A-11C show a proximal handle for a dilator.

FIG. 12 is a side elevational partial cross section of a catheter having a cannulated guide rail extending therethrough over a guidewire.

FIG. 13 is a cross sectional view through a dual dilator system.

FIGS. 14A-14B show an example of a blood reintroduction system.

FIGS. 15A-15C show another example of a blood reintroduction system.

FIG. 16 shows another example of a blood reintroduction system.

FIG. 17 shows another example of a blood reintroduction system.

FIGS. 18A-18B show another example of a blood reintroduction system.

FIG. 19A shows an example of a canister for use with a blood reintroduction system.

FIG. 19B shows an example of a filter for use with a blood recirculation system.

FIG. 19C shows an example of a filter lock for use with a filter and a blood recirculation system.

FIGS. 20A-20C show an example of a canister for use with a blood reintroduction system.

FIGS. 21A-21B show an example of a filter housing for use in a filter assembly.

FIGS. 22A-22B show an example of a cap for use in a filter assembly.

FIGS. 22C-23 show the cap shown in FIGS. 22A-22B attached to a canister.

FIG. 24A shows another example of a canister for use with a blood reintroduction system.

FIG. 24B shows an example of a downstream filter system that can be used with the canister of FIG. 24A.

FIG. 25 shows an exploded view of the canister of FIG. 24A.

FIG. 26 shows an example of a vacuum console with the canister removed.

FIG. 27 shows an example of a canister with a first and second filter for use with a blood reintroduction system.

FIG. 28 shows another example of a canister for use with a blood reintroduction system.

FIG. 29 shows another example of a canister for use with a blood reintroduction system.

FIG. 30 shows another example of a canister for use with a blood reintroduction system.

FIG. 31 shows another example of a blood reintroduction system.

### DETAILED DESCRIPTION

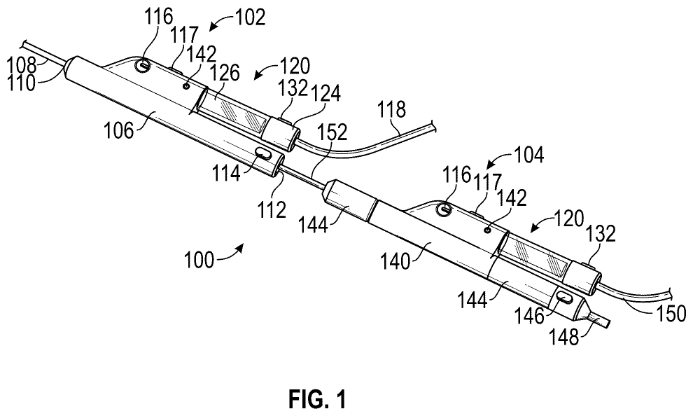

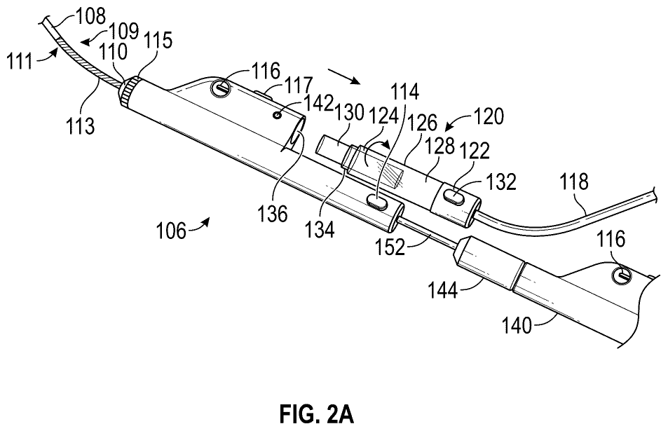

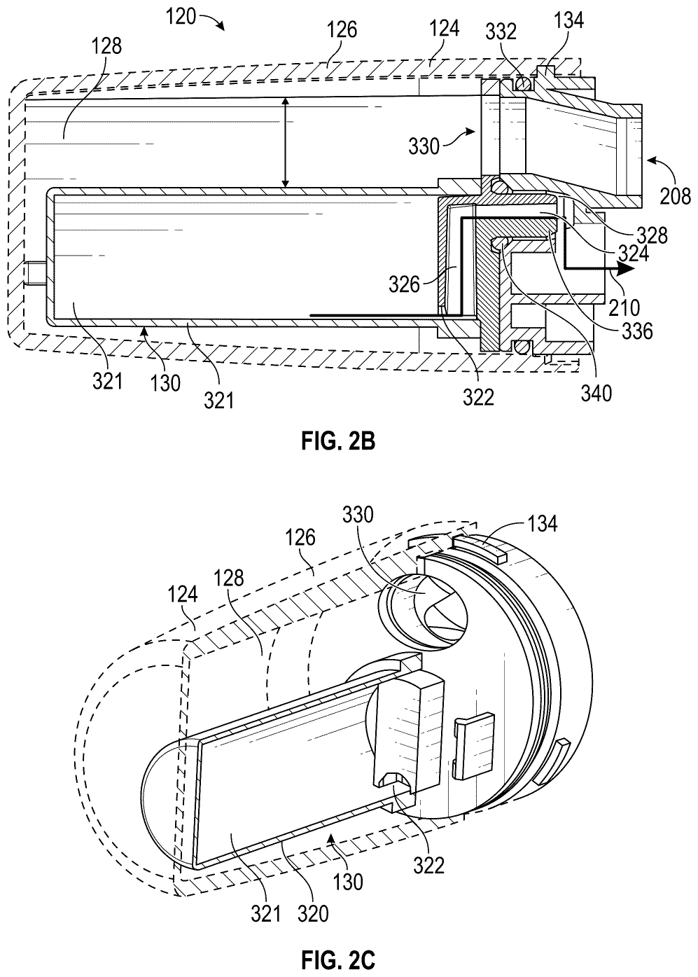

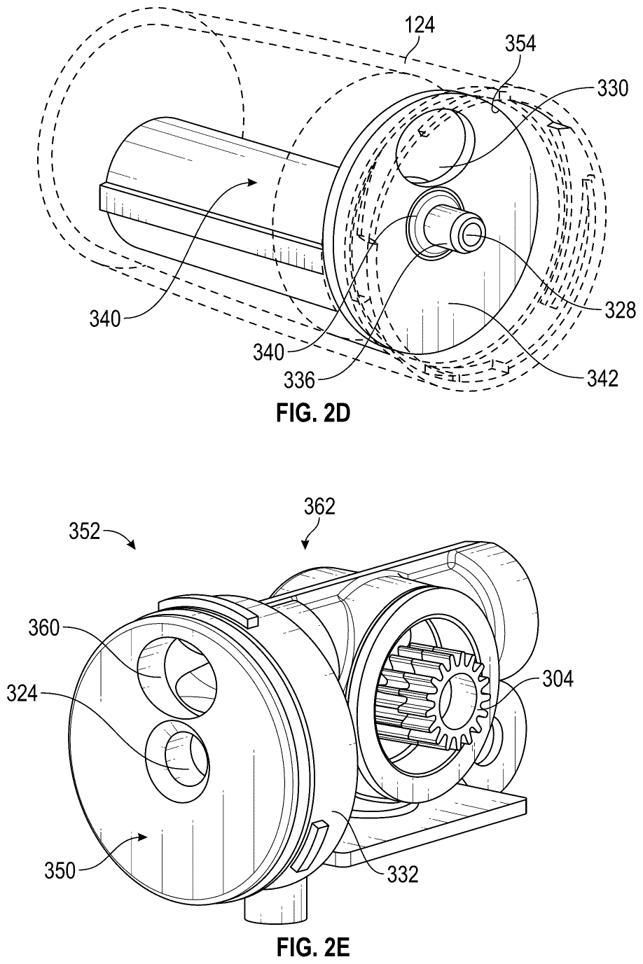

Referring to FIGS. 1 and 2A, there is illustrated a further implementation of an aspiration system 100. The system includes a first thrombectomy catheter 102, such as a large bore aspiration catheter, and a second aspiration catheter 104 which is optionally advanceable through the first thrombectomy catheter 102 as has been discussed, or used by itself.

Thrombectomy catheter 102 comprises a proximal handle 106 having an elongate flexible tubular catheter body 108 extending distally therefrom. The proximal end 110 of the tubular body 108 may be permanently carried by the proximal handle 106 or may be provided with a releasable connector for detachable connection to a complementary connector on the handle 106.

In one implementation, the tubular body 108 or 152 or both are provided with a flexible neck 109 extending between proximal end 110 and a transition 111. The flexible neck 109 has a greater flexibility than the adjacent portion of the tubular body 108 distal to the transition 111. The flexible neck 109 may have a length of at least about 2 cm and often at least about 4 cm, but generally no more than about 20 cm or 10 cm or less.

The sidewall of the catheter body 108 within flexible neck 109 includes a helical coil 113 having adjacent filars spaced apart to both improve flexibility, and also allow visualization between adjacent windings of the coil. At least the flexible neck 109 includes a sidewall window such as the spaces between adjacent coil windings which may be in the form of an optically transparent outer tubular layer, such as any of a variety of optically transparent shrink tubing polymers. This allows visualization of clot through the side wall as it passes through the neck 109 before it enters the proximal handle. The transparent window on the larger catheter 108 also allows visualization of the distal tip of the inner catheter 152 as it passes the window. This may be facilitated by placing a visual marker on the distal end of the inner catheter 152 such as a colored annular band.

For example, in an implementation having a 24 French tubular body 108, the smaller tubular body 152 (e.g. 16 French catheter) may be provided with a visual indicium such as a white tip on the distal end, that can be visualized through the sidewall window as it passes through the flexible neck 109. The flexible neck 109 may also be provided on the catheter shaft 152.

The spring coil 113 may extend distally to a point of termination within about one or 2 cm of the transition 111, and, and one implementation, at the transition 111. Distally of the transition, the sidewall of tubular body 108 may include a tubular braid, importing greater stiffness and higher push ability than the helical coil 113.

The proximal end of the catheter may be provided with a rotation control such as a rotatable knob 115 which may be rotationally fixed to the catheter and rotatable with respect to the handle housing. This facilitates relative rotation between the catheter and the housing for any of the large or small bore catheters disclosed herein.

A central lumen extending through the tubular catheter body 108 is in communication with a flow path extending through the proximal handle 106 to a proximal access port 112. The flow path between the tubular catheter body 108 and the proximal access port 112 is preferably linear, to axially movably receive the second catheter 104 which may or may not be utilized in a given procedure. To accommodate the absence of second catheter 104 and seal the port 112, the proximal handle 106 is preferably provided with a homeostasis valve 114 such as a Thuohy-Borst valve.

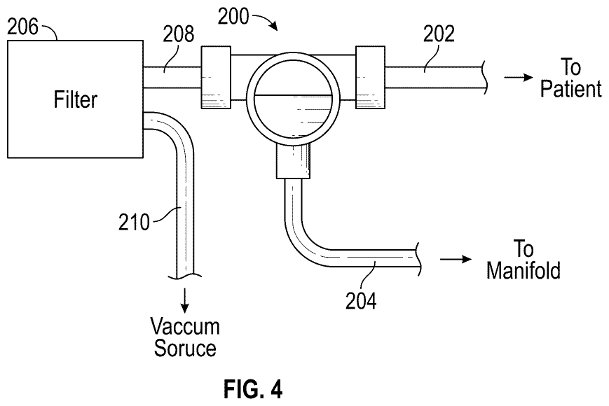

A manifold switch 116 controls two way or three way a manifold valve (illustrated in FIG. 4) for selectively controlling fluid flow as discussed further below. An aspiration control 117 is provided to turn aspiration on and off. Alternatively, manifold switch 116 can be configured to turn aspiration one and off.

A filter assembly 120 includes housing 122 with a side wall 124, at least a portion of which includes a transparent window 126. Window 126 permits a viewing of the contents (e.g. aspirated clot) of a filter chamber 128, which contains a filter 130.

The filter assembly 120 is configured to place the filter 130 in the flow path between the tubular catheter body 108 and the aspiration tubing 118. Preferably the filter chamber can be closed to maintain negative pressure conveyed from a pump via aspiration tubing 118, or opened to permit insertion or removal of the filter 130. In the illustrated implementation, the filter assembly 120 is removably connected to the handle 106. A connector 134 such as a first thread on the housing 122 is releasably engageable with a complementary connector 136 such as a complementary thread on the handle 106. A vent (aperture) to atmosphere may be provided in communication with the filter chamber, to reduce foaming of blood in response to reduced pressure.

An implementation may include an integrated flow control module in the proximal handle 106. Thus, an adjustable flow regulator (not illustrated) may be positioned in the flow path, to enable controllable toggling of the aspiration between a low flow mode and a high flow mode. In the illustrated implementation, optional flow regulator is positioned downstream of the filter 130, and contained within the housing 122 of the filter assembly 120. A flow regulator control 132 is provided, to control the flow rate. Preferably, as has been discussed, the flow regulator is configured to regulate fluid flow through the flow path at a default low flow rate. Activation of the flow control 132 adjust the flow to the high flow rate mode. Flow control 132 may be a momentary button, slider switch, trigger, knob or other structure that is preferably defaulted to the low flow mode.

In any of the catheters disclosed herein, carrying the filter chamber 128 on the catheter or at least spaced apart from the remote vacuum pump and vacuum canister provides enhanced aspiration performance. The location of a conventional aspiration pump may be far enough away from the patient to require a length of aspiration tubing between the pump and the catheter to be as much as 50 inches or 100 inches or more. The pump typically includes an aspiration canister for blood collection. When aspiration is desired, a valve is opened to place the low pressure canister in communication with the catheter by way of the aspiration tubing, to aspirate material from the patient. But the length of the aspiration tubing operates as a flow restrictor, causing a delay between the time of activating the vacuum button and actual application of suction to the clot.

In some embodiments, the catheter handle 106 or 140 contains a filter chamber 128 for example, which is in communication with the vacuum canister on the pump by way of elongate aspiration tubing 118. The momentary aspiration control 117 is in between the filter chamber 128 and the catheter, which, in the default off position, allows the entire length of the aspiration tubing 118 and the filter chamber 128 to reach the same low pressure as the aspiration canister on the pump. The flow restriction between the pump canister 129 and the filter chamber 128 is greater than the flow restriction between the filter chamber 128 and the patient.

In an alternate configurations, 117 may be a vent to atmosphere which allows the clot canister to be evacuated. Element 142 can alternatively be an injection port such as for injecting contrast media, saline, or drugs.

Thus, the only remaining flow restrictor between a source of vacuum (filter chamber 128) and the patient is the relatively short aspiration pathway between the valve in the proximal handle and the distal end of the catheter. When the momentary aspiration control 117 is activated, the flow restriction and enclosed volume on the patient side of the filter chamber is low relative to the flow restriction and enclosed volume through aspiration tubing 118 on the pump side of the filter chamber 128.

This dual chamber configuration produces a rapid spike in negative pressure experienced at the distal end of the catheter upon activation of the aspiration control 117. The response time between activating the aspiration control 117 and realizing suction actually experienced at the clot is significantly faster and allows significantly higher initial flow than the response time realized in a conventional system having only a vacuum chamber located at the pump.

The spike of negative pressure experienced at the distal end of the catheter will fade as pressure equilibrium is reached between the filter chamber and canister. When the momentary aspiration control 117 is closed, the vacuum pump will gradually bring the pressure in the filter chamber 128 back down to the level in the vacuum canister at the pump.

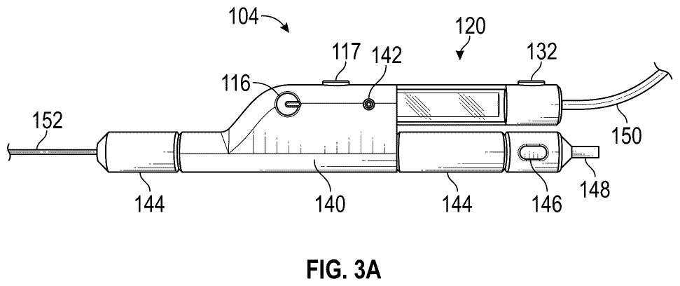

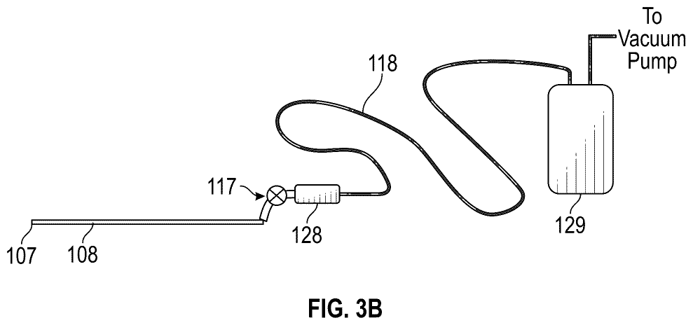

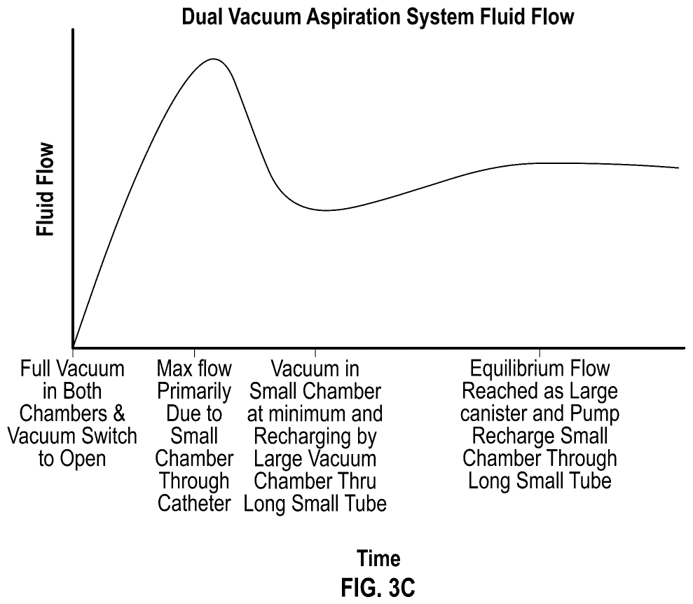

A simplified fluid flow diagram is illustrated in FIG. 3B, and a qualitative flow rate diagram is illustrated in FIG. 3C. The flow restriction between chamber 128 and the distal and 107 of catheter 108 is small relative to the flow restriction between the vacuum canister 129 and the vacuum chamber 128. This allows a negative pressure peak experienced at distal end 107 almost instantaneously upon activation of vacuum switch 117. The flow rate of material into the catheter 108 rapidly reaches a peak and subsides as vacuum chamber 128 fills with aspirated material. The vacuum in chamber 128 declines to a minimum, and slowly recharges by the large vacuum chamber 129 and associated pump through tubing 118. In use, a clinician may choose to allow the momentary vacuum switch 117 to close at or shortly following the maximum flow rate, just giving a short burst or spike of vacuum to facilitate spiration of thrombus into the catheter 108.

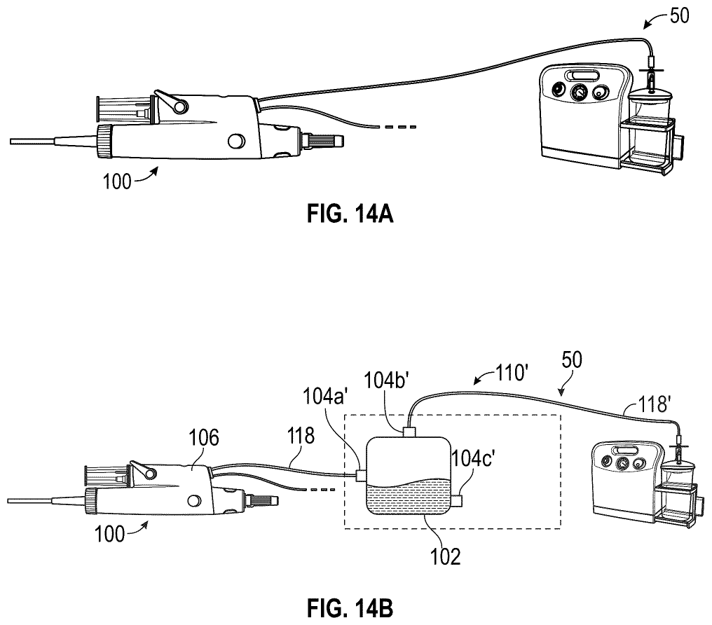

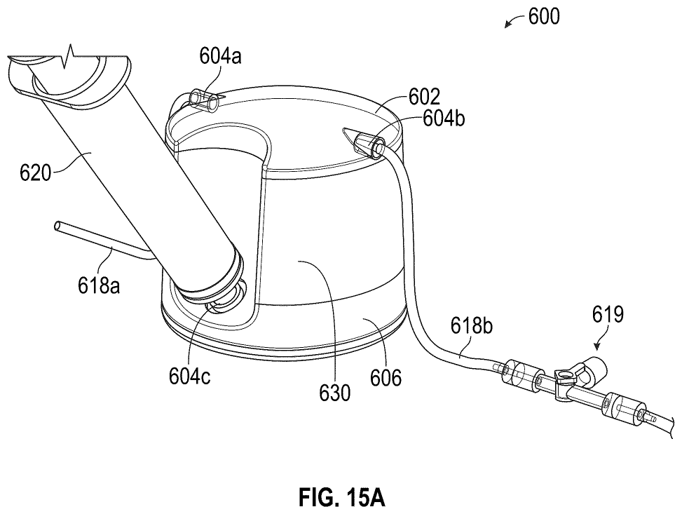

FIGS. 14A-31 illustrate views of various examples of blood reintroduction systems. It will be understood that any of the features, structures, materials, methods, or steps that is described and/or illustrated in any embodiment in any of the blood reintroduction systems of any one of FIGS. 14A-31 can be used with or instead of any feature, structure, material, method, or step that is described, illustrated, and/or contemplated herein.

A blood reintroduction system 110′ can be positioned between the aspiration system 100 and the aspiration pump 50, as shown in FIG. 14B. The blood reintroduction system 110′ can include a canister 102′ having one or more ports in fluid communication with the aspiration system 100 and/or the aspiration pump 50. For example, the canister 102′ can include an inlet 104a′, a first outlet 104b′, and a second outlet 104c′. In some cases, the canister 102′ and the aspiration system 100 are in fluid communication via the aspiration tubing 118. One end of the aspiration tubing 118 can be connected to the handle 106, which is described in relation to FIGS. 1-2A, and the other end can be connected to, for example, the inlet 104a′. The canister 102′ and the aspiration pump 50 can be in fluid communication via tubing 118′. One end of the tubing 118′ can be connected to, for example, the first outlet 104b′ and the other end can be connected to the aspiration pump 50, which is described in relation to FIG. 1-8. In some cases, the port 104c′ can act as an extraction port for clinicians to extract blood collected inside the canister 102′. Blood can be extracted from the canister 102′ via the second outlet 104c′ using, for example, a syringe. The aspiration pump 50 can provide aspiration via the tubing 118′ and the tubing 118. The aspiration can facilitate collection of blood and debris inside the canister 102′. In some cases, the blood reintroduction system 110′ can be positioned within a sterile field.

To facilitate the extraction of blood at the second outlet 104c′ (e.g., the extraction port) and to prevent air from being drawn into the syringe, the canister 102′ can include a tapering bottom portion. For example, the interior bottom portion of the canister 102′ can taper such as conically, narrow end towards the bottom portion, or otherwise inclined, to improve the flow of blood towards a low point of the canister 102′ in communication with the second outlet 104c′. Beneficially, this can improve the concentration of blood at or near the second outlet 104c′ allowing clinicians to extract blood from the canister 102′ via the second outlet 104c′. The canister 102′ can also include one or more support structures for maintaining an upright position or securing the canister to a patient and/or pole/bedrail. In some cases, the canister 102′ can include a downwardly extending skirt and/or legs to allow the canister 102′ to sit on a surface and remain in its upright position so that the outlet 104c′ communicates directly with the lowest point of the canister 102′. The body of the canister 102′ or support structures removably or permanently carried by the canister 102′ can also be shaped to conform to the shape of a patient's body. For example, a base of the canister 102′ can be contoured to sit across the top of a patient's legs. In some cases, the canister can include one or more support structures, such as a hook, for removably placing the canister on an IV pole and/or bedrail.

In some cases, the blood reintroduction system 110′ can include a flop tube (not shown). The flop tube can include weight on one end which can cause that end to gravitate towards a typically hemispherical bottom interior wall of the canister 102′ regardless of the orientation of the canister 102′. The weight of the flop tube can beneficially place the opening of the flop tube in contact with a bottom portion of the canister 102′ thus ensuring that the syringe draws fluids from a bottom a bottom portion of the canister 102′ and preventing the syringe from drawing air.

Blood extracted from the canister 102′ via the second outlet 104c′ can be reintroduced to a patient. For example, blood extracted by the extraction device, such as the syringe, can be injected directly or indirectly to a patient. In some cases, the blood can be injected into the vasculature of a patient. The blood can also be injected into a patient from the syringe to, for example, a venous line connected to a patient. In some cases, the blood reintroduction system 110′ can include a venous line (not shown) connected to a patient on one end and to the second outlet 104c′ on the other end. The venous line can beneficially facilitate fluid reinfusion into a patient by drawing blood collected inside the canister 102′ to the patient via the venous line. In such cases, it may not be necessary for clinicians to manually extract blood from the canister 102′ and reinfuse the blood to the patient. That is, blood collected inside the canister 102′ can be automatically reinfused by flowing though the venous line into the vasculature of a patient or a flow path into a vasculature of patient. The venous line can be in fluid communication with a pump to facilitate blood reinfusion by flowing blood from the canister 102′ to the patient.

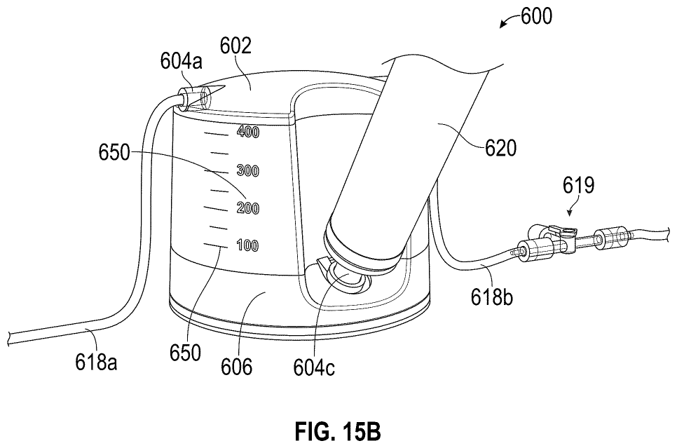

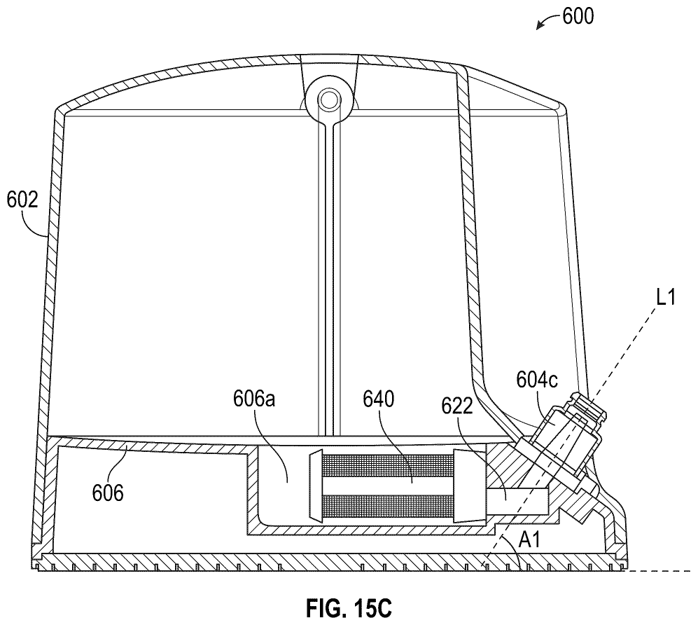

FIGS. 15A-15C show an example of a blood reintroduction system. The blood reintroduction system 600 can include a canister 602, an inlet 604a, a first outlet 604b, a second outlet 604c, and a base 606. The canister 602 can include graduations to easily visualize and/or measure the amount of blood collected within the canister 602. For example, the canister 602 can include one or more graduations 650 which can beneficially provide an indication of the amount of fluid 630 (e.g., blood) collected inside the canister 602. The inlet 604a and the first outlet 604b can be connected to other medical devices via tubing. For example, a first aspiration tubing 618a can be connected to the inlet 604a, and a second aspiration tubing 618b can be connected to the first outlet 604b. The first aspiration tubing 618a can place the canister 602 and an aspiration system, such as aspiration system 100 (as described in relation to FIG. 1) in fluid communication with each other. The first and second aspiration tubing 618a, 618b can include high pressure braided tubing. In some cases, the blood reintroduction system 600 can be positioned within a sterile field.

Blood drawn into the canister 602 via the first aspiration tubing 618a can collect on a bottom portion of the canister 602. The second aspiration tubing 618b can place an upper air space within the canister 602 and an aspiration pump, such as aspiration pump 50 (as described in relation to FIGS. 1-8) in fluid communication with each other. The vacuum generated by the aspiration pump 50 can beneficially facilitate aspiration of blood from the first aspiration tubing 618a to the canister 602. Although reference is made to the second aspiration tubing 618b being in fluid communication with an aspiration pump, the second tubing 618b can be in communication with any vacuum source. For example, the second tubing 618b can be in communication with a syringe. In operation, the negative pressure exerted by the syringe can facilitate aspiration via the first tubing 618a.

The interior surface of the canister 602 can include a coating to provide one or more of a variety of properties to the canister 602. In some instances, the coating may be configured to enhance visualization through at least a portion of the canister 602. The coating may be configured to inhibit blood accumulation or increase blood repellant properties. In some instances, the canister 602 may comprise a coating to inhibit foam formation during an aspiration procedure. The coatings may be located at least partially along an interior surface of the canister 602. The coating can be both hydrophobic and oleophobic. In some instances, the coating may have some hydrophilic features on a portion of the polymer to increase oleophobic properties.

The first aspiration tubing 618a and the second aspiration tubing 618b can include one or more valves. For example, the first aspiration tubing 618a and/or the second aspiration tubing 618b can include one or more stopcock valves 619. Opening and/or closing the stopcock valve 619 along the first aspiration tubing 618a and/or the second aspiration tubing 618b can beneficially allow users to control aspiration and/or the flow of blood through the first aspiration tubing 618a and/or the second aspiration tubing 618b. Opening the stopcock 619 can open the first aspiration tubing 618a and/or the second aspiration tubing 618b to atmosphere to facilitate withdrawal of blood by allowing venting of atmospheric air into the canister 602 regulate and/or equalize pressure while withdrawing blood via a syringe 620.

In some cases, the inlet 604a and the first outlet 604b can be positioned on a top portion of the canister 602. The inlet 604a and the first outlet 604b can be positioned on opposite ends of the canister 602. Positioning the first outlet 604b on a top portion of the canister 602 can beneficially prevent the first outlet 604b, which is in fluid connection with an aspiration pump, such as aspiration pump 50 which is described in relation to FIGS. 1-8, from aspirating fluid 630 and instead allow the fluid 630 to collect inside the canister 602. The second outlet 604c can be positioned on or in communication with a bottom portion of the canister 602 such as the lowest point in the chamber to optimize blood removal. In some cases, the second outlet 604c can include a luer fitting. The second outlet 604c can receive a medical device, such as the syringe 620. For example, a syringe can be used to extract fluid 630 (e.g., blood) collected inside the canister 602 by inserting the syringe 620 inside the second outlet 604c and extracting the fluid 630. To prevent fluid 630 from escaping the canister 602 via the second outlet 604c, the second outlet 604c can include a self-closing valve such as a displaceable membrane which obstructs the outflow path unless displaced such as by the distal tip of the syringe coupled to the outlet 604c. The membrane can be disposed inside the second outlet 604c and block the flow of liquid from an interior of the canister 602 to the exterior.

The syringe 620 can be used to extract fluid 630 from the canister 602 via the second outlet 604c. This can beneficially allow users to withdraw fluids 630 (e.g., blood) collected inside the canister 602 for infusion into a patient without significant, or any, interruption in the aspiration procedure and/or any disconnection of any components of the blood reintroduction system 600. That is, a physician or user may simply engage the syringe 620 to the second outlet 604c at any point during the aspiration procedure to withdraw blood from the canister 602 and reintroduce the blood to the patient. This can allow blood to be withdrawn from the canister 602 while still applying aspiration to the canister 602 and/or any of the devices and/or components in fluid communication with the canister 602 (e.g., the first tubing 618a, the inlet 604a, an aspiration catheter, etc.). Positioning of the blood reintroduction system 600 within a sterile field can significantly reduce the risk of blood contamination prior to reintroduction of the blood into the patient.

Fluid 630 extracted from inside the canister 602 via the second outlet 604c can be reintroduced to a patient. For example, the fluid 630 extracted by the extraction device, such as the syringe 620, can injected directly or indirectly to a patient. In some cases, the fluid 630 can be injected into the vasculature of a patient. The fluid 630 can also be injected to a patient from the syringe 620 to, for example, a venous line connected to a patient. In some cases, the blood reintroduction system 600 can include a venous line (not shown) connected to a patient on one end and to the second outlet 604c on the other end. The venous line can beneficially facilitate fluid reinfusion into a patient by drawing fluid 630 collected inside the canister 602 to the patient via the venous line. In such cases, it may not be necessary for clinicians to manually extract fluid 630 from the canister 602 and reinfuse the fluid 630 to the patient. That is, fluid 630 collected inside the canister 602 can be automatically reinfused by flowing though the venous line into the vasculature of patient or a flow path into a vasculature of patient. Reinfusion may be assisted by a pump in between the outlet 604c and the patient (not illustrated).

In some cases, the blood reintroduction system 600 can include one or more filters. The one or more filters can be positioned upstream of the second outlet 604c, at the second outlet 604c, at the syringe 620, and/or along a flow path (e.g., a venous line between the second outlet 604c and the patient) before reinfusion of the fluid 630 to the patient. In some cases, the one or more filters can be positioned between a downstream opening of the second outlet 604c and an upstream internal space of the canister 602.

For example, the blood reintroduction system 600 can include a filter 640, as shown in in FIG. 15C. The filter 640 can be positioned inside a cavity 606a of the base 606. In some cases, a shape and/or dimensions of the cavity 606a are larger than a shape and/or dimensions of the filter 640 to allow the cavity 606a to receive and secure the filter 640. The filter 640 can prevent solid material, such as a blood clot and/or thrombus, collected inside the canister 602 from reaching the second outlet 604c. This can beneficially prevent solid matter from being extracted via the second outlet 604c using an extraction device, such as the syringe 620. The filter 640 can trap solid matter while allowing fluids, such as fluid 630 (e.g., blood), to flow through the filter 640. In some cases, a cavity 622 is positioned between the filter 640 and the second outlet 604c, as shown in FIG. 15C. The space between the filter 640 and the second outlet 604c provided by the cavity 622 can collect the fluid 630 exiting the filter 640 (e.g., filtered blood). The filter 640 can beneficially allow filtering of the blood prior to introduction of the blood into the patient.

Although reference is made to the filter 640 being positioned inside the cavity 606a of the base 606, the filter 640 can be positioned anywhere along a flow path extending from the inlet 604a to the second outlet 604. In some cases, the filter 640 can be positioned anywhere between the canister 602 and the vasculature of the patient. In some cases, the blood reintroduction system 600 can include more than one filter 640. For example, the system 600 can include a first filter positioned distal to the inlet 604a and a second filter proximal to the inlet 604b. The first and second filters can include the same or different filter ratings. For example, the first filter can include pores larger than the pores of the second filter. This can prevent clots and/or particles larger than a porosity of the first filter from reaching the canister 602. The use of two or more filters can beneficially prevent any solid material, such as a blood clots and/or thrombus, from reaching the second outlet 604c. In some cases, the first filter can be part or otherwise positioned within the handle 106 of the aspiration system 100 (as described in relation to FIG. 1), and the second filter can include the filter 640 (as described in relation to FIGS. 15A-15C). This configuration, in some instances, may advantageously permit pre-filtered blood to into the blood reintroduction system 600 to provide an additional filtration layer prior to blood withdrawal and reintroduction into a patient.

In some cases, a longitudinal axis of the second outlet 604c can incline from a plane defined by the base 606 at an angle. For example, a longitudinal axis L1 of the second outlet 604c and the base 606 can form an angle A1, as shown in FIG. 15C. In some cases, the angle A1 can be an acute angle. For example, the angle A1 can be between about 10° and about 80°. For example, the angle A1 can be between about 10° and about 40°, between about 20° and about 50°, between about 30° and about 60°, between about 40° and about 70°, or between about 50° and about 80°. In some cases, the angle can be about 45°. The angle A1 can ensure that the extraction mechanism (e.g., the needle) of the syringe 620 can connect to a bottom portion of the canister 602, such as the low point in cavity 622 without mechanical interference from the sidewall of the canister 602. This can beneficially allow the syringe 620 to draw fluid 630 collected on a bottom portion of the canister 602 and prevent the syringe 620 from drawing air.

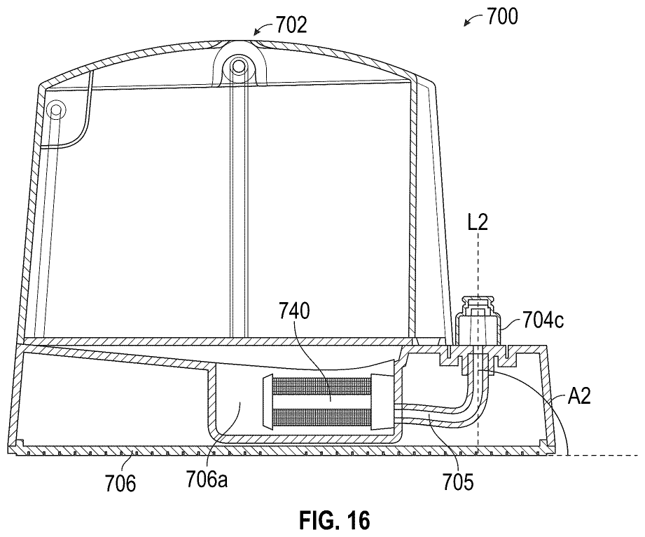

FIG. 16 shows another example of a blood reintroduction system. Like the blood reintroduction system 600, the blood reintroduction system 700 can include a canister 702, an inlet, a first outlet, a second outlet 704c, and a base 706. The inlet and the first outlet can be connected to other medical devices via tubing. For example, a first aspiration tubing can be connected to the inlet, and a second aspiration tubing can be connected to the first outlet. The first aspiration tubing can place the canister 702 and an aspiration system, such as aspiration system 100 (as described in relation to FIG. 1) in fluid communication with each other. Blood drawn into the canister 702 via the first aspiration tubing can collect on a bottom portion of the canister 702. The second aspiration tubing can place the canister 702 and an aspiration pump, such as aspiration pump 50 (as described in relation to FIGS. 1-8) in fluid communication with each other. The vacuum generated by the aspiration pump 50 can beneficially facilitate aspiration of blood from the first aspiration tubing to the canister 702.

Like the blood reintroduction system 600, the blood reintroduction system 700 can include one or more filters. The one or more filters can be positioned upstream of the second outlet 704c, at the second outlet 704c, at a syringe, and/or along a flow path (e.g., a venous line between the second outlet 704c and the patient) before reinfusion of the fluid to the patient. In some cases, the one or more filters can be positioned between an outlet opening of the second outlet 704c and an internal volume of the canister 702. For example, the blood reintroduction system 700 can include a filter 740. The filter 740 can be positioned inside a cavity 706a of the base 706. In some cases, a shape and/or dimensions of the cavity 706a are larger than a shape and/or dimensions of the filter 740 to allow the cavity 706a to receive and secure the filter 740. The filter 740 can prevent solid matter, such as thrombus, collected inside the canister 702 from reaching the second outlet 704c. This can beneficially prevent solid matter from being extracted via the second outlet 704c using an extraction device, such as a syringe. The filter 740 can trap solid matter while allowing fluids (e.g., blood), to flow past the filter 740. In some cases, the second outlet 704c can include an extension tube 705 in fluid communication with the filter 740. The extension tube 705 can be sealed to one end of the filter 740, as shown in FIG. 16. A lumen inside the extension tube 705 can direct the fluid exiting the filter 740 (e.g., filtered blood) to the second outlet 704c.

In some cases, the second outlet 704c can extend from a base 706 at an angle. For example, a longitudinal axis L2 of the second outlet 704c and the base 706 can form an angle A2, as shown in FIG. 16. Unlike the angle A1 of the blood reintroduction system 600, the angle A2 of the blood reintroduction system 700 can include a right angle. For example, the angle A2 can be between about 90° and about 45°, between about 90° and about 75°, or between about 80° and about 70°. The angle A2 can ensure that the syringe can couple to the outlet port on extension tube 705 without mechanical interference from the canister 702. This can beneficially allow the syringe to draw filtered fluid through the extension tube 705 and prevent the syringe from drawing air.

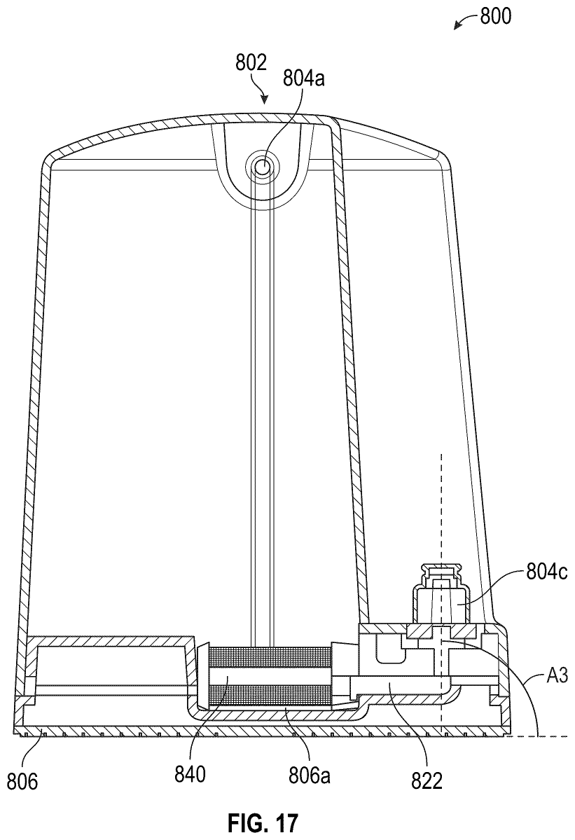

FIG. 17 shows another example of a blood reintroduction system. Like the blood reintroduction systems 600 and 700, the blood reintroduction system 800 can include a canister 802, an inlet 804a, a first outlet, a second outlet 804c, and a base 806. The inlet 804a and the first outlet can be connected to other medical devices via tubing. For example, a first aspiration tubing can be connected to the inlet 804a, and a second aspiration tubing 818b can be connected to the first outlet. The first aspiration tubing can place the canister 802 and an aspiration system, such as aspiration system 100 (as described in relation to FIG. 1) in fluid communication with each other. Blood drawn into the canister 802 via the first aspiration tubing can collect on a bottom portion of the canister 802. The second aspiration tubing can place the canister 802 and an aspiration pump, such as aspiration pump 50 (as described in relation to FIGS. 1-8) in fluid communication with each other. The vacuum generated by the aspiration pump 50 can beneficially facilitate aspiration of blood from the first aspiration tubing to the canister 802.

Like the blood reintroduction systems 600 and 700, the blood reintroduction system 800 can include one or more filters. The one or more filters can be positioned upstream of the second outlet 804c, at the second outlet 804c, at a syringe, and/or along a flow path (e.g., a venous line between the second outlet 804c and the patient) before reinfusion of the fluid to the patient. In some cases, the one or more filters can be positioned between a downstream opening of the second outlet 804c and an internal volume of the canister 802. For example, the blood reintroduction system 800 can include a filter 840. The filter 840 can be positioned inside a cavity 806a of the base 806. In some cases, a shape and/or dimensions of the cavity 806a are larger than a shape and/or dimensions of the filter 840 to allow the cavity 806a to receive and secure the filter 840. The filter 840 can prevent solid matter, such as thrombus, collected inside the canister 802 from reaching the second outlet 804c. This can beneficially prevent solid matter from being extracted via the second outlet 804c using an extraction device, such as a syringe. The filter 840 can trap solid matter while allowing fluids (e.g., blood), to flow past the filter 840. Like the blood reintroduction system 600, the blood reintroduction system 800 can include a cavity 822 positioned between the filter 840 and the second outlet 804c, as shown in FIG. 17. The space between the filter 840 and the second outlet 804c provided by the cavity 822 can collect the fluid exiting the filter 840 (e.g., filtered blood).

In some cases, a longitudinal axis L3 of the second outlet 804c and a base 806 can form an angle A3. Like the angle A2 of the blood reintroduction system 700, the angle A3 of the blood reintroduction system 800 can include a right angle relative to the plane of the base. For example, the angle A3 can be between about 90° and about 45°, between about 90° and about 75°, or between about 80° and about 70°. The angle A3 can ensure that the extraction mechanism (e.g., the needle) of the syringe can couple to the second outlet 804c on the cavity 822 without mechanical interference from the canister 802. This can beneficially allow the syringe to draw filtered fluid through the cavity 822 and prevent the syringe from drawing air.

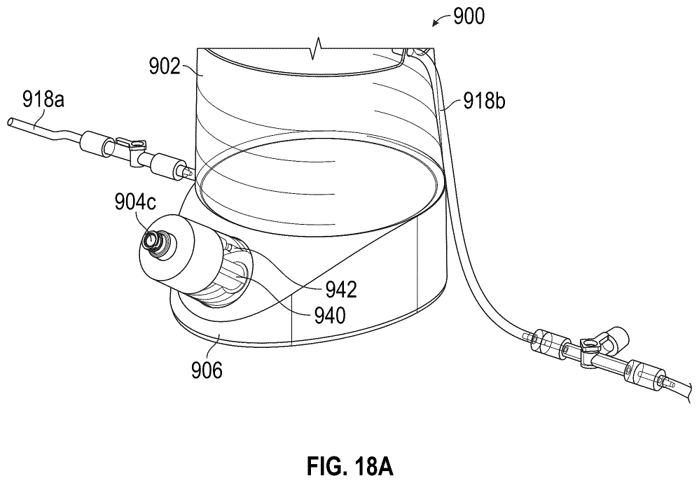

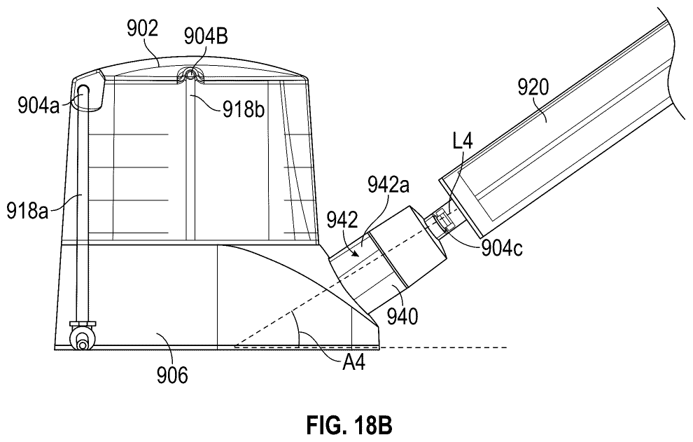

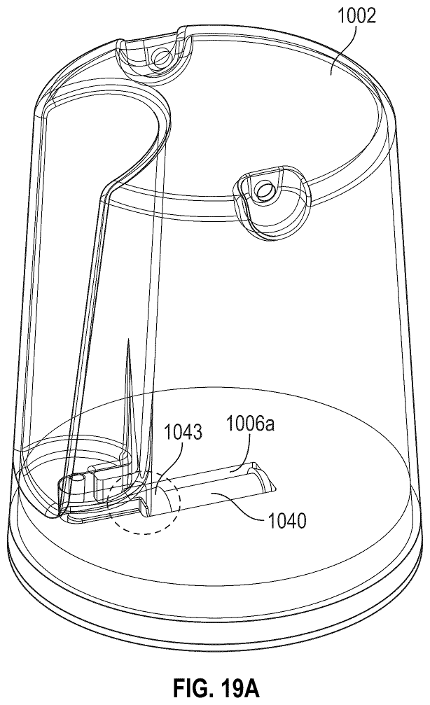

FIGS. 18A-18B show another example of a blood reintroduction system. Like the blood reintroduction systems 600, 700, and 800, the blood reintroduction system 900 can include a canister 902, an inlet 904a, a first outlet 904b, a second outlet 904c, and a base 906. The inlet 904a and the first outlet 904b can be connected to other medical devices via tubing. For example, a first aspiration tubing 918a can be connected to the inlet 904a, and a second aspiration tubing 918b can be connected to the first outlet 904b. The first aspiration tubing 918a can place the canister 902 and an aspiration system, such as aspiration system 100 (as described in relation to FIG. 1) in fluid communication with each other. Blood drawn into the canister 902 via the first aspiration tubing 918a can collect on a bottom portion of the canister 902. The second aspiration tubing 918b can place the canister 902 and an aspiration pump, such as aspiration pump 50 (as described in relation to FIGS. 1-8) in fluid communication with each other. The vacuum generated by the aspiration pump 50 can beneficially facilitate aspiration of blood from the first aspiration tubing 918a to the canister 902.

Like the blood reintroduction systems 600, 700, and 800, the blood reintroduction system 900 can include one or more filters. The one or more filters can be positioned upstream of the second outlet 904c, at the second outlet 904c, at a syringe 920, and/or along a flow path (e.g., a venous line between the second outlet 904c and the patient) before reinfusion of the fluid (e.g., blood) to the patient. In some cases, the one or more filters can be positioned between an opening of the second outlet 904c and an internal space of the canister 902. For example, the blood reintroduction system 900 can include a filter 940. Unlike the filters 640, 740, and 840, of the blood reintroduction systems 600, 700, and 800, the filter 940 of the blood reintroduction system 900 can be positioned outside the canister 902. For example, the filter 940 can be positioned inside a canister connector 942. The canister connector 942 can extend from the base 906. The canister connector 942 can include a window 942a, such as a transparent side wall, to permit visualization of any debris trapped on an upstream surface of the filter membrane. In some cases, a shape and/or dimensions of the canister connector 942 are larger than a shape and/or dimensions of the filter 940 to allow the canister connector 942 to receive and secure the filter 940. The filter 940 can prevent solid matter, such as thrombus, collected inside the canister 902 from reaching the second outlet 904c. This can beneficially prevent solid matter from being extracted via the second outlet 904c using an extraction device, such as the syringe 920. The filter 940 can trap solid matter while allowing fluids, such as fluid (e.g., blood), to flow past the filter 940.

In some cases, a longitudinal axis L4 of the second outlet 904c and/or the canister connector 942, and the base 606 can form an angle A4, as shown in FIG. 18B. Like the angle A1 of the blood reintroduction system 600, the angle A4 of the blood reintroduction system 900 can be between about 10° and about 80°. For example, the angle A4 can be between about 10° and about 40°, between about 20° and about 50°, between about 30° and about 60°, between about 40° and about 70°, or between about 50° and about 80°.

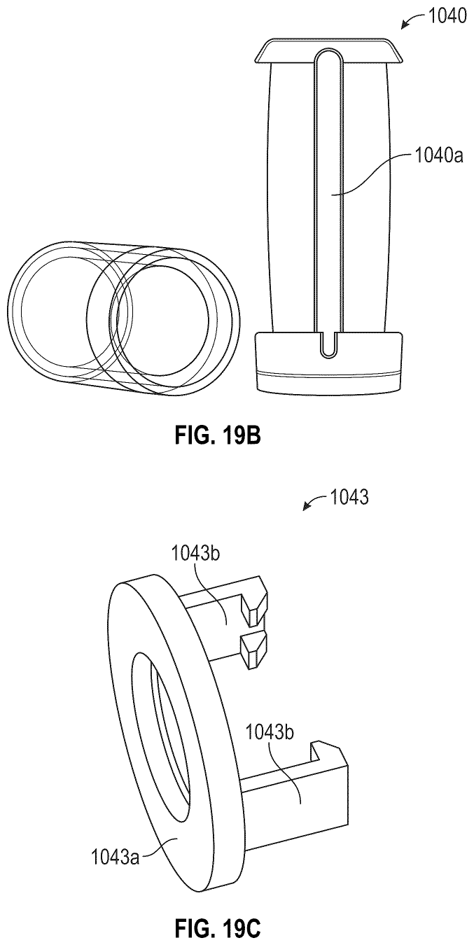

FIG. 19A shows an example of a canister 1002 for use with a blood reintroduction system (e.g., blood reintroduction systems 600, 700, 800, 900). A securing mechanism can be used to secure a filter, such as filters 640, 740, 840, inside a canister. For example, a filter lock 1043 can be used to secure a filter 1040, which is shown in FIG. 19B and which can be identical or similar to filters 640, 740, 840, and/or 940), inside the cavity 1006a. The filter 1040 an include a support element 1040a extending across a length of the filter 1040. The support element 1040a can beneficially prevent collapse of the filter when vacuum is applied. The filter 1040 can be a substantially planar porous membrane, or tubular (e.g., cylindrical) as shown in FIG. 19B, with an upstream surface on the outside and a downstream surface on the inside. The filter lock 1043 can include a cap 1043a and one or more arms 1043b extending from the cap, as shown in FIG. 19C. In some cases, the arms 1043b can extend from the cap 1043a perpendicularly. The cap 1043a can be secured to the cavity 1006a and the arms 1043b can secure the filter 1040. The filter lock 1043 can beneficially secure the filter 1040 inside the cavity 1006a and prevent the filter 1040 from moving inside the canister 1002.

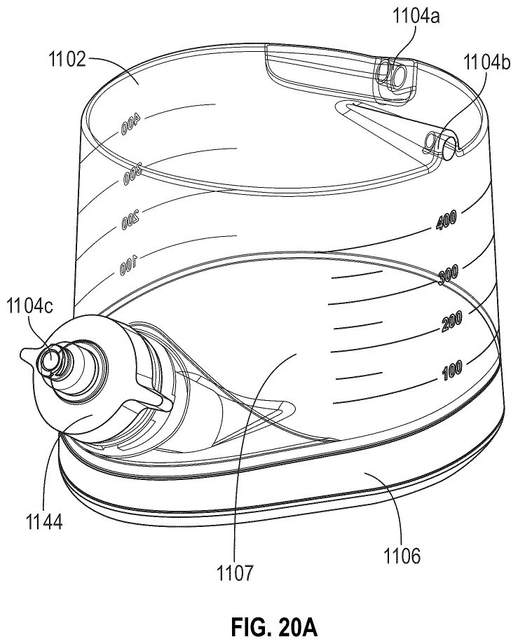

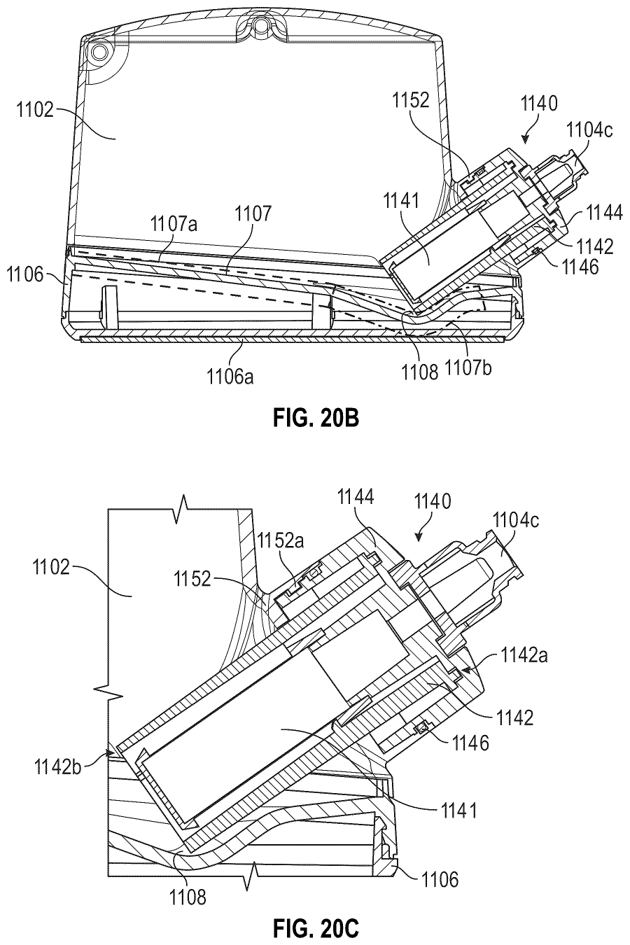

FIGS. 20A-20C show another example of a canister 1102. The canister 1102 can be used, for example, in a blood reintroduction system, such as blood reintroduction systems 600, 700, 800, 900, 100 which are described in relation to FIGS. 15A-18B. The canister 1102 can include an inlet 1104a, a first outlet 1104b, a second outlet 1104c, all in communication with a chamber defined by the canister 1102, and a base 1106. In some cases, the base 1106 can form a floor 1107 exposed to an interior volume of the canister 1102. In some cases, the floor 1107 can include one or more portions. For example, the floor 1107 can include a first portion 1107a and a second portion 1107b. The first portion 1107a can be inclined to facilitate flowing of the fluids (e.g., blood) collected inside the canister 1102 towards the second portion 1107b. The second portion 1107b may be the lowest point in the enclosed volume of the canister 1102 and can define a dip 1108. In some cases, a lower portion 1106a of the base 1106 can include a friction enhancing surface structure and/or material to prevent the canister 1102 from moving and-or slipping. The lower portion 1106a can include a silicone rubber coating, layer, and or feet which can beneficially provide a gripping surface.

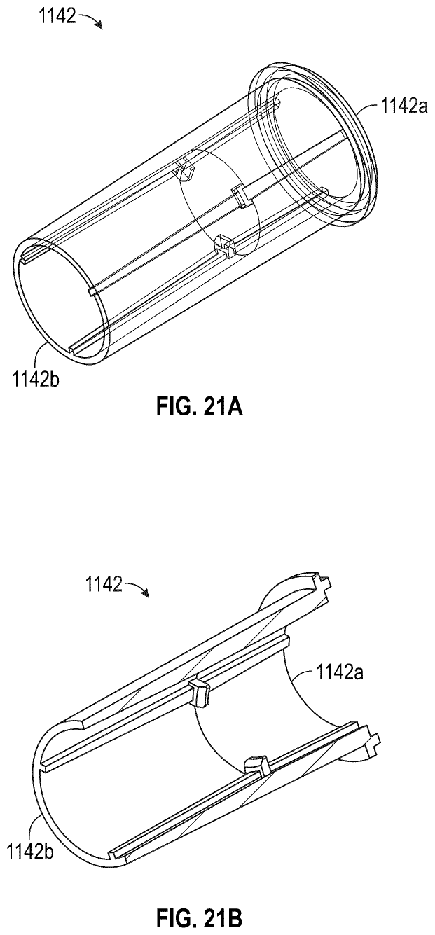

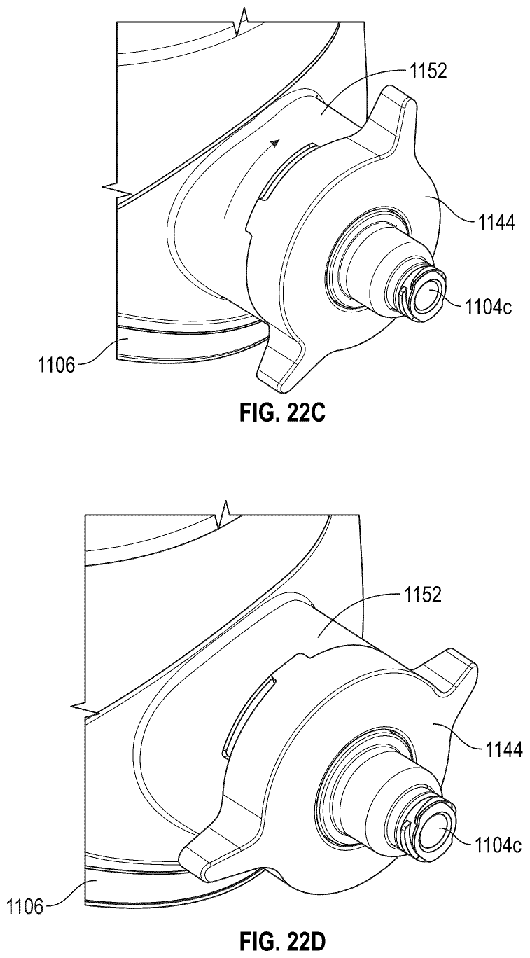

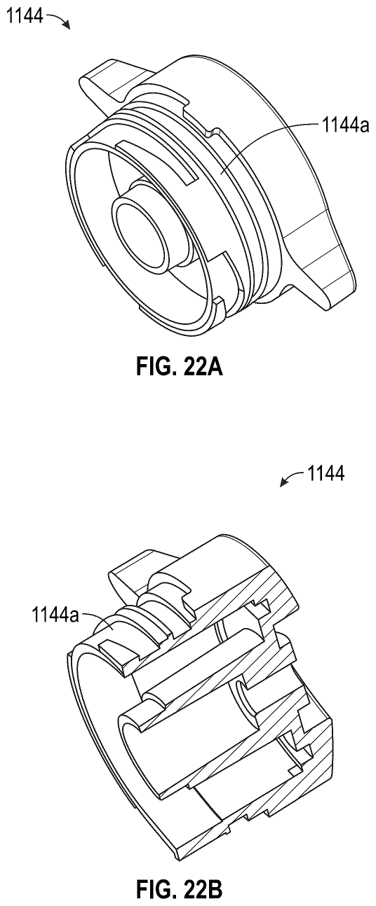

A filter assembly 1140 is carried by and can be positioned at least partially inside the canister 1102. For example, the filter assembly 1140 can be positioned inside a canister connector 1152 at least partially formed by the canister 1102. In some cases, the canister connector 1152 can extend from the canister 1102. The base 1106 and the canister connector 1152 can form an acute angle similar or identical to angels A1 and/or A4, which are described in relation to FIGS. 15C and 18B respectively. As shown in FIGS. 21A-22B, the filter assembly 1140 can include a filter housing 1142 and a cap 1144. The filter housing 1142 can include a proximal end 1142a and a distal end 1142b. The cap 1144 can be attached to the proximal end 1142a of the filter housing 1142. In some cases, the cap 1144 can be attached to the filter housing 1142 using a threaded engagement or adhesive.

The filter assembly 1140 can also include a filter 1141. The filter 1141 can be positioned inside the filter housing 1142, as shown in FIGS. 20A and 20B. The filter 1141 can include a membrane including a 10, 15, 20, 30, 40, 50, 100, and/or a 200-micron rating. The filter can include a membrane including a rating of at least 100 microns. In some cases, the filter 1141 includes a cylindrical side wall defining a hollow interior. The filter 1141 can include a membrane wrapped around the hollow center of the cylindrical scaffold.

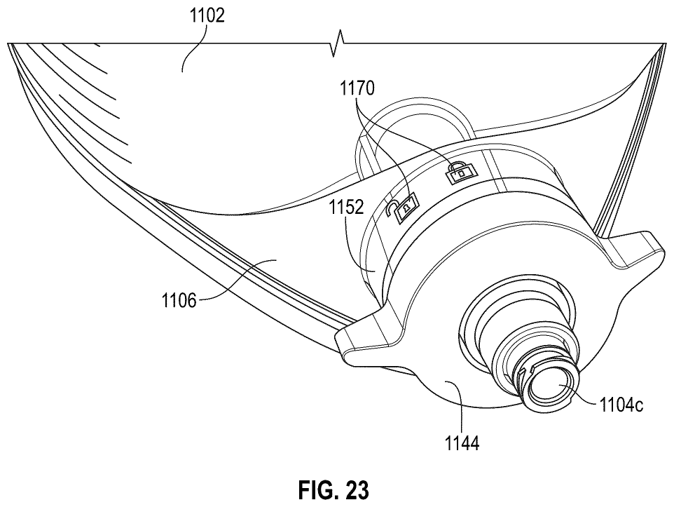

The cap 1144 can include a thread 1144a, as shown in FIGS. 22A and 22B, which can facilitate attachment of the filter assembly 1140 to the canister connector 1152. An exterior portion of the canister connector 1152 can include a thread 1152a. The thread 1152a can receive the thread 1144a of the cap 1144 thus securing the cap 1144 to the canister connector 1152. The cap 1144 can be secured to the canister connector 1152 by, for example, rotating the cap 1144 from a first, unlocked position, as shown in FIG. 22C, to a second, locked position, as shown in FIG. 22D, thereby securing the threads 1152a, 1144a to each other. An O-ring 1146 can be positioned between the cap 1144 and the canister connecter 1152 to create a seal between the cap 1144 and the canister connecter 1152 when the cap 1144 is secured to the canister connector 1152. In some cases, the canister connector 1152 and/or the cap 1144 can include one or more visual and/or tactile elements 1170, as shown in FIG. 23, for indicating whether the cap 1144 is in the unlocked position (e.g., not secured to the canister connector 1152) and/or a locked position (e.g., secured to the canister connector 1152).

While the cap 1144 and the canister connector 1152 are illustrated with a threaded interlocking system, it will be understood that the cap 1144 and the canister connector 1152 may incorporate any suitable interlocking mechanism (e.g., an adhesive, snap-fit, interference fit, etc.) to engage the cap 1144 with the canister connector 1152. The interlocking mechanism can beneficially allow physicians and/or users to remove and replace the filter assembly 1140 and/or the filter 1141 if, for example, the filter 1141 gets clogged during use.

In some cases, the second outlet 1104c can include a valve such as a luer fitting. The second luer fitting can be secured to the cap 1144. Blood can be extracted from the canister 1102 via the luer fitting using an extraction device such as a syringe. The syringe can be similar or identical to syringes 620, and/or 920. The filter 1141 can prevent solid matter, such as thrombus, collected inside the canister 1102 from reaching the luer fitting. This can beneficially prevent solid matter from being extracted via the luer fitting using the extraction device. The filter 1141 can trap solid matter while allowing fluids (e.g., blood), to flow through the filter 1141 and into the extraction device.

In embodiments where the second outlet 1104c includes a luer fitting, the luer fitting can allow the second outlet 1104c to receive and secure other luer fitting. For instance, a syringe with a luer fitting can be connected and secured to the luer fitting of the second outlet 1104c. The luer fitting can beneficially allow for the precise control of withdrawal of fluids from the canister 1102. The luer fitting can also provide a secure connection to the syringe and/or tubing and prevent leaks at the second outlet 1104c. The luer fitting can include different sizes and dimensions to accommodate different syringes and/or tubing.

The filter 1141 allows absorption of fluids and extraction of the fluids using the extraction device (e.g., syringe) when the filter 1141 is in fluid contact with the fluids inside the canister 1102. To improve and maintain contact between the filter 1141 and the fluids collected inside the canister 1102, the distal end 1142b of the filter housing 1142 and the distal end of the filter 1141 can positioned at least partially in the low spot such as dip 1108 formed by the second portion 1107b of the floor 1107. The inclined surface of the first portion 1107a can facilitate flow of the fluids toward the dip 1108 formed by the second portion 1107b and allow the distal tip of the filter 1141 to contact the bottom-most part of the floor 1107. This can beneficially enhance contact between the distal tip of the filter 1141 and the fluids, thus allowing extraction of the fluids using the extraction device and preventing users from extracting air. The inclined first portion 1107a can allow any blood inside the canister 1102 to be immediately directed towards the dip 1108 where the filter 1141 is closest to the floor 1107.

A bottom edge of the filter 1141 can be transverse to a longitudinal axis of the filter housing 1142. In some cases, once the filter 1141 gets wet (e.g., the filter 1141 contacts the blood collected inside the canister 1102), it can absorb the blood collected in the canister 1102. This can beneficially allow the extraction device, such as a syringe, to withdraw blood from the filter 1141 even when only a portion of the filter 1141 is in contact with the blood inside the canister 1102. The proximity of the distal end of the filter 1141 to the dip 1108 reduces the amount of blood required for the filter 1141 to first contact the blood. In some cases, as little as 50 cc of blood may collect inside the dip 1108 before the blood contacts the distal end of the filter 1141.

A flow path extending between the dip 1108 and the second outlet 1104c can allow blood to collected at or near the dip 1108, flow through the filter 1141, and exit the canister 1102 via the second outlet 1104c. Physicians and/or users can extract blood from the canister 1102 using the syringe as long as any part of the filter 1141 is in contact with the blood collected in the dip 1108. The syringe can draw the blood absorbed by the filter 1141 through the sidewalls of the filter 1141 until the blood is drawn into the syringe via the second outlet 1104c. As blood is withdrawn using the syringe, any remaining blood inside the canister 1102 can flow to the dip 1108 where it can be absorbed by the filter 1141 and/or subsequently withdrawn using the syringe. This can beneficially prevent the blood from settling inside a portion of the canister 1102 where the blood may be prevented from being withdrawn using the syringe.

In any of the embodiments disclosed herein, blood can flow into the canister 1102 through an inlet aligned a top portion of the canister 1102. The blood can wash along the sidewall of the canister 1102. This can allow physicians and/or users to easily identify the presence of blood inside the canister 1102. The sidewalls of the canister 1102 can provide a smooth pathway (e.g., decrease sheer stress and/or improve laminar flow) for the blood to flow towards the floor 1107 which can beneficially prevent and/or decrease hemolysis. For example, the inlet 1104a can be oriented so that blood is directed to an interior of the sidewall of the canister 1102 as blood flows from the aspiration catheter into the canister 1102. This can allow a circumferential blood flow along the inter of sidewall. The circumferential blood flow can result in the blood flowing along the sidewall before eventually collecting on the floor 1107 of the canister 1102. The circumferential blood flow can beneficially minimize turbulence and/or splashes. In some cases, the flow of blood exiting the inlet 1104a can form a tangent relative to the interior of the sidewall.

Additional details of the filter assembly and related structures are illustrated in FIGS. 2B to 2E. Referring to FIG. 2B, the filter assembly 120 includes a tubular sidewall 124 having a transparent window 126. In some implementations the entire tubular sidewall 124 can be a transparent window. The side wall 124 encloses a filter 130 as has been discussed. The filter 130 includes a tubular filter sidewall 320 defining an interior chamber 321 for filtered blood. Filtered blood is drawn in the direction of vacuum line 210 through a first vacuum aperture 322 and into a flow path 324 having a vertical offset 326 in the flow path 324. The vertical offset 326 allows removal of blood from the bottom of the chamber, through a flow path and out through a second vacuum aperture more centralized with respect to a central axis of the tubular sidewall 124 and in communication with vacuum line 210.

The filter 130 is displaced downward with respect to a central longitudinal axis of the tubular sidewall 124, leaving the filter chamber 128 having a chamber height 129 at least as great as the inside diameter of a filter line aperture 330 leading to filter line 208. This allows clot to move from filter line 208 into the filter chamber 128 without restriction, and optimizes the volume of filter chamber 128 on top of the filter 130 for viewing through the window 126.

A connector 134 maybe carried by the filter assembly 120, such as in the form of a bayonet mount, or other releasable attachment to the proximal handle housing. A first seal 332 such as an annular elastomeric ring may be provided between the tubular sidewall 124 and the complementary surface on the proximal handle housing.

A second vacuum aperture 328 is in communication with the first vacuum aperture 322 by way of the flow path 324. Second vacuum aperture 328 may be carried on an axially extending tubular projection 336 which may be removably received within a complementary recess on the proximal handle housing.

A second seal 340 such as an elastomeric ring maybe provided surrounding the flow path 324, for providing a seal between the filter assembly and the proximal handle. In the illustrated implementation, the second seal 340 surrounds the tubular projection 336 and is configured to seal against an adjacent complementary surface on the proximal handle in the as mounted orientation.

Referring to FIG. 2D, the filter assembly 120 additionally includes a filter base 342 through which filter line aperture 330 extends. The flow path 324 additionally extends through the filter base 342, and, in the illustrated implementation, exits the tubular projection 336 carrying the second vacuum aperture 328.

A complementary docking platform 350 is carried by the proximal handle, having complementary connector to connector 134 for rapid attachment and detachment of the filter assembly 120 from the proximal handle. In the illustrated embodiment, at least a first flange 352 may be received through an opening 354 on the filter assembly 120. Rotation of the filter assembly 120 moves the first flange into interference fit with a second flange 356 to secure the filter assembly 120 to the docking platform 350 on the proximal handle. Two or three or four or more similar flange and complementary opening pairs may be provided around the periphery of the components. In the illustrated implementation, the circumferential arc length of the flange and corresponding opening on one of the three pairs is greater than the other two pairs to function as a key, so that the filter assembly can only be secured to the docking platform in a single rotational orientation.

The docking platform 350 includes a filter line aperture 360 for communicating with filter line 208, and a vacuum line aperture 362 for placing the filter 130 in communication with a source of vacuum. The docking platform 350 may be connected to a two way valve 362 or a three way valve as is discussed elsewhere herein depending upon the desired functionality. The valve may carry a rotatable drive gear 304 to rotate the interior rotatable valve gate as is discussed in additional detail below. Alternatively, a lever or other control on the housing may be configured to rotate a shaft directly coupled to the rotatable part of the valve.

A valved flow path may also be provided for venting the filter chamber 128 directly to atmosphere. The valve may be opened such as by depressing a momentary button, which is biased in the closed direction. This can create an abrupt change in pressure at the distal end of the catheter, which may facilitate clot aspiration. This can also be used to discharge vacuum

Referring to FIG. 3A, additional details of the handle 140 of the second catheter 104 are disclosed. The handle 140 extends between a proximal end and a distal end. An elongate flexible tubular body 152 extends distally from the distal end of the handle 140 and is configured to advance distally through the proximal handle 106 and the tubular body 108 of thrombectomy catheter 102.

A steering dial 144 may be provided to place one or more steering wires under tension, to deflect a deflection zone near the distal end of the tubular body 152. A manifold switch 116 may be provided to control the flow of fluid as will be discussed below. The handle additionally comprises an aspiration control 117 such as a slider switch, for turning aspiration on or off. A max button 132 may be provided for delivering a momentary pulse of high aspiration rate as has been discussed.

Fluid flow through the thrombectomy system is controlled by manifold switch 116 (see, e.g., FIG. 1), which may control a two way or three-way valve. Referring to FIG. 4, a schematic flow diagram for three-way valve 200 is provided. Patient line 202 can be placed in fluid communication with the patient, via a catheter such as a large diameter thrombectomy catheter 12 or second catheter 42.

Patient line 202 may be placed in communication with a manifold line 204 by advancing the three-way valve 200 to a first position, such as to allow delivery of medications, contrast media or saline to the patient.

Adjustment of the three-way valve 200 to a second position can isolate patient line 202 and place the manifold in communication with the filter 206 via filter line 208. Activation of a vacuum pump will draw blood from the patient and through the filter 206 via vacuum line 210.

Further adjustment of the three-way valve 200 to a third position will place the manifold in communication with the vacuum line 210, such as to permit a saline flush of the filter 206. This third position may be eliminated depending upon the desired functionality.

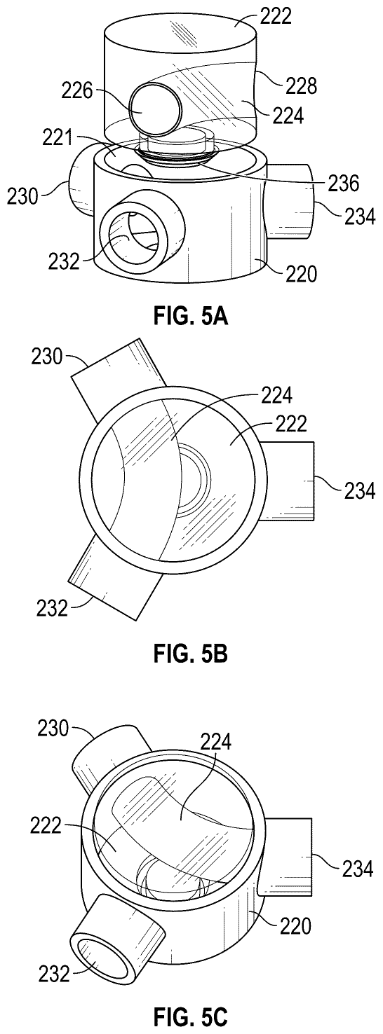

One implementation of a suitable three-way valve 200 is illustrated in FIGS. 5A through 5C. Referring to FIG. 5A, the valve 200 may comprise a housing 220 such as a cylindrical housing having a central cavity 221. A rotatable cylindrical gate 222 may be positioned in the central cavity 221, as illustrated in the exploded view of FIG. 5A. Rotatable gate 222 is provided with a flow path 224 extending between a first end 226 and a second end 228. In the illustrated implementation, the first end 226 and a second end 228 of the flow path are spaced apart around the circumference of the rotatable gate by approximately 120 degrees.

In the rotational orientation of the rotatable gate 222 illustrated in FIG. 5A, the first end 226 of the flow path 224 is in communication with a first port 232, and the second end 228 of the flow path 224 is in communication with a second port 234. This corresponds to the first position discussed previously, in which the patient is in fluid communication with the manifold.

FIG. 5B illustrates rotatable gate 222 in the second position where the flow path 224 places the first port 232 in communication with the third port 230 to place the filter 206 in communication with the manifold. The rotatable gate 222 may be toleranced within the cavity 221 such that the rotatable gate 222 seals the second port 234 thus isolating the patient from the flow path in this orientation. Similarly, in each of the other two orientations, two of the ports are placed in communication with the flow path, while the third port is isolated from the flow path.

The third position is illustrated in FIG. 5C, in which the flow path places the second port 234 in communication with the third port 230, placing the filter 206 in communication with the patient, and isolating the manifold from the flow circuit.

The foregoing selectivity may be achieved by spacing the three ports approximately 120 degrees apart around the circumference of the housing, to cooperate with the flow channel 224 end ports which are about 120 degrees apart around the circumference of the cylindrical gate 222. The gate 222 may be rotated within the housing 220 by a connector 236 extending through the housing 220 such as along the axis of rotation, and connected to a control 116 such as a rotatable knob, lever or slider switch with a rack and pinion drive assembly.

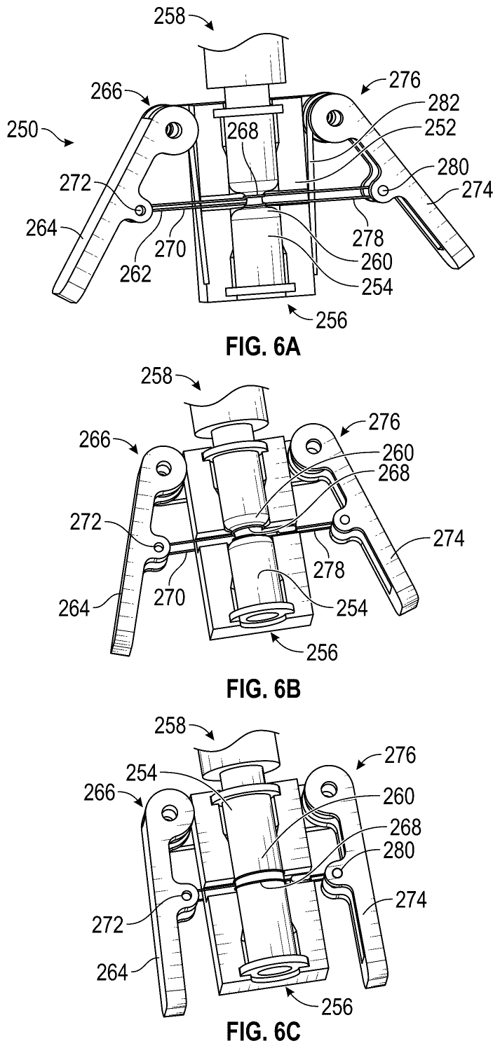

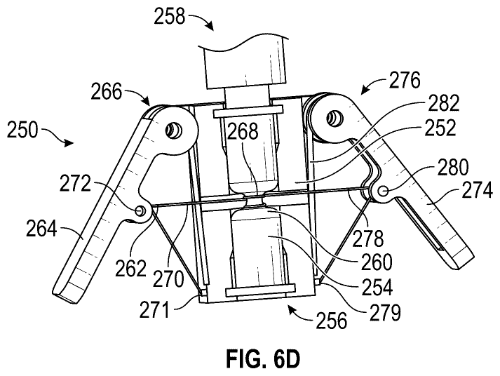

Each of the catheters disclose herein may be provided with a hemostasis valve on the proximal end, to allow selective closing of the central lumen to completely closed without any devices extending therethrough, from a sealed fit around devices of differing diameters such as a guide wire or a secondary catheter extending therethrough. One example of a suitable hemostasis valve is schematically illustrated in FIGS. 6A through 6C.

Referring to FIG. 6A, hemostasis valve 250 includes a frame 252 for supporting a flow path defined within a tubular sidewall 254. The frame 252 may be integrally formed with or mounted to the catheter handle or hub.

The flow path and tubular sidewall 254 extend between a first end 256 and a second end 258. First end 256 may be a port 112 (see, e.g., FIG. 1) on the proximal end of any of the catheters disclosed herein. Second end 258 may be in communication with the central lumen of the corresponding aspiration catheter, such that devices entering the first end 256 and advanced axially through the flow path can advance all the way to the distal end of the aspiration catheter and beyond.

At least a portion 260 of the sidewall 254 is collapsible in response to external pressure. That portion 260 and optionally the full length of the tubular sidewall within valve 250 may be comprise a collapsible elastic tube such as silicone tubing, which is biased into an open lumen tubular configuration when unconstrained. A compression element such as filament 262 is configured to apply compressive force against the sidewall 254 to reduce the inside diameter of the flow path to provide a seal against itself (when completely closed with no devices extending therethrough) or against a device such as a guidewire or catheter extending therethrough. In the illustrated implementation, the filament 262 forms a loop 268 around the collapsible portion 260 of tubular sidewall 254. Retraction of a first tail portion 270 of the filament 262 away from the sidewall 254 constricts the diameter of the loop 268 thereby collapsing the portion 260 of the tubular sidewall as illustrated in FIG. 14A.

In the illustrated implementation, the first tail portion 270 of the filament 262 may be retracted by at least a first lever 264. Lever 264 may be connected to the frame 252 by a first pivot 266 and is attached to the tail portion 270 at an attachment point 272. Advance of the lever in a first direction places the filament under tension and reduces the inside diameter of the valve. Releasing the lever removes the tension and the collapsible portion 260 of the sidewall rebounds to its unconstrained, open lumen configuration.

In the illustrated implementation, a second lever 274 is attached to the frame 252 at a second pivot 276, and is attached to a second tail portion 278 of the filament 262. Each of the first and second tail portions may comprise a single filament or two or three or more parallel filaments. In the two filament configuration as illustrated, the filaments may be immovably secured to the lever, or may be a continuous filament, looped around a fulcrum 280. The loop 268 may comprise one or two or three or more revolutions around the tubular sidewall, depending upon the desired performance.

At least one lever 264 is provided with a spring 282 to bias the lever away from the tubular sidewall, constricting the inside diameter of the collapsible portion 260 into sealing engagement with a device extending therethrough, or to a completely closed configuration in the absence of a device. As illustrated, a second lever 274 may also be biased using the same spring or a second spring.

As illustrated in FIG. 6C, compression of the levers in a medial direction towards the axis of the tubular sidewall 254 releases tension on the tail portions of the filament and allows the valve to open, such as to permit advance of a catheter through the valve. Releasing the levers allows the spring bias to retract the tail portions, reducing the diameter of the loop 268 and collapsing the collapsible portion 260 into sealing engagement with the outside surface of the secondary catheter, at an intermediate valve diameter as seen in FIG. 6B.

Retraction of the tail portion 270 of filament 262 may alternatively be accomplished by winding the tail portion 270 around a rotatable spool such as a shaft or drum. Rotation of a knob or advance of a lever causes the spool to take up filament and collapse the sidewall.

An alternate configuration for the filament 262 is illustrated in FIG. 14 D. In this implementation, the first tail portion 270 slidably extends around a first fulcrum at 272 and returns to attach to the housing at an attachment point 271. First tail portion 270 extends from the fulcrum to form a loop 268 around the collapsible tube. The filament 262 may make a single revolution or two or more revolutions around the collapsible tube before continuing on around a second fulcrum at 280, to a second point of attachment 279 to the housing.

Compression of the first lever 264 and second lever 274 loosens the loop 268, allowing the lumen to resume patency. Releasing the levers allows the spring bias to reduce the diameter of the loop 268 as the first tail portion 270 and second tail portion 278 slide away from each other around the left and right fulcrums. Preferably, friction between the filament 262 and fulcrums are minimized, as by providing a lubricious oil such as silicone oil around the fulcrums at 280 and 272, as well as using Teflon braided line for the filament 262.

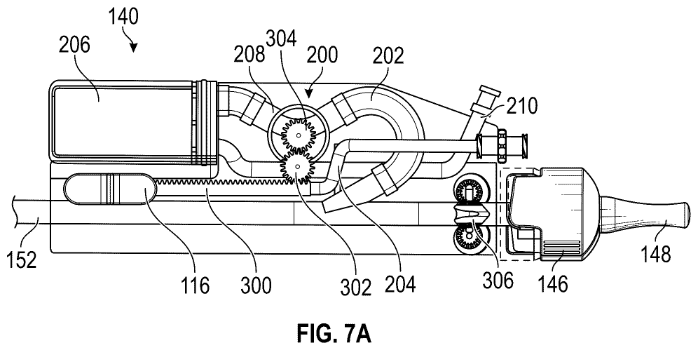

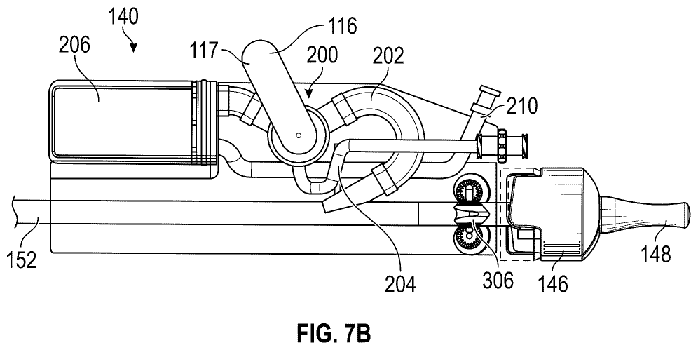

Various components of the aspiration system handle are schematically represented in context in FIG. 7A. The proximal handle 140 on a second catheter 104 includes a filter 206, a tubular body 152 and other features previously described. Two-way or three-way valve 200 selectively controls flow among the filter line 208, patient line 202 and manifold line 204. In this implementation, the three-way valve control 116 is in the form of the slider switch. The slider switch axially movably displaces a first linear rack gear 300. Rack gear 300 engages a pinion gear 302, which may either directly rotate the gate in the valve 200, or, as illustrated, drive a third gear 304 which rotates the rotatable gate within 200. An alternative valve control system is schematically illustrated in FIG. 15 B. In this implementation, the slider switch, linear rack gear 300 and pinion gear 302 omitted. A valve control 116 in the form of a lever 117 is attached directly to a shaft which controls rotation of the valve gate. The lever may be advanced proximally or distally, to adjust the flow path through the valve as has been discussed.

A steering mechanism 306 is provided to permit steering of the second catheter 152. Manually rotatable knob 148 allows manual rotation of a core wire and distal helical tip as has been discussed. The core wire axially movably extends across hemostasis valve 146. Alternatively, the core wire and tip (e.g., thrombus engagement tool 400) may be coupled to a motorized drive unit at the proximal end of the catheter system.

In certain implementations, an aspiration catheter such as a 16 French catheter is advanced transvascularly over a wire and/or through a larger diameter (e.g., 24 French aspiration catheter) to the treatment site. If the application of vacuum is not able to aspirate the clot into the 16 French catheter, an elongate flexible thrombus engagement tool may be advanced through the 16 French aspiration catheter, to facilitate retrieval of the clot.

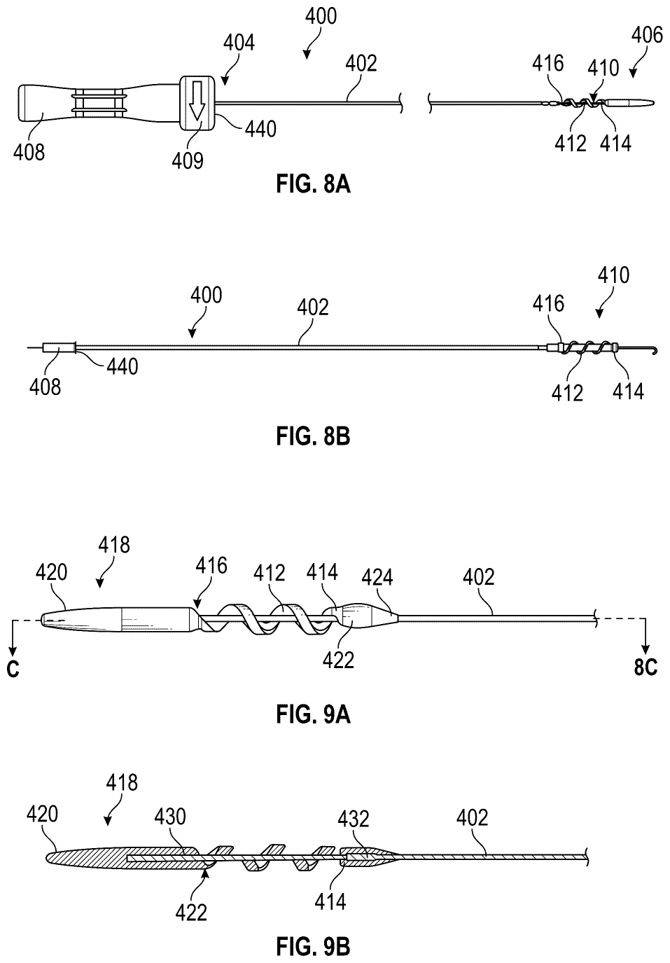

Referring to FIGS. 8A and 8B, the thrombus engagement tool 400 may comprise an elongate flexible shaft 402 having a proximal end 404 and a distal end 406. A proximal handle such as a handle 408 may be configured to be rotated by hand. Distal end 406 carries a clot engagement tip 410 which may include one or more radially outwardly extending structures such as a helical thread 412. The handle 408 may have an indicium of rotational direction such as a printed or molded arrow 109 which indicates the direction to rotate the handle 408 in order for the helical thread 412 to engage clot.

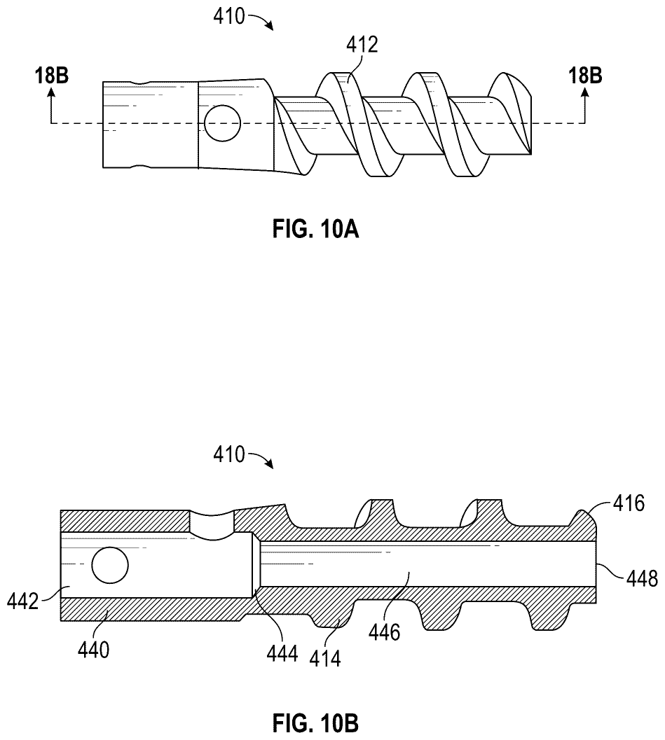

In one implementation illustrated in FIG. 8B, the thrombus engagement tool 400 carries a clot engagement tip 410 of the type illustrated in FIGS. 10A and 10B. The proximal end of the tip 410 is glued to the distal end of a braid-reinforced polyimide tube. The proximal end of the Microlumen has a cannulated torquing handle 408, and the whole assembly is cannulated so it can be delivered and function over a wire 468 such as an 0.035″ wire. The 0.035″ wire helps maintain space between the tip and the vessel wall, and the wire can be pulled back inside the working length of the flexible shaft 402 during rotation and engagement with the clot as needed.

Referring to FIG. 9A, the distal tip 410 includes a helical thread 412 extending from a distal end 414 to a proximal end 416 and supported by flexible shaft 402. The axial length of the distal tip 410 is at least about 2 mm or 5 mm or 10 mm and in some embodiments no more than about 30 mm or 20 mm measured along the flexible shaft 402. The helical thread 412 wraps around the axis at least about 1 or 2 or 4 or more full revolutions, but in some embodiments no more than about 10 or 6 revolutions. In some embodiments the axial length along the threaded portion of the tip is within the range of from about 1 to about 8 revolutions.

The helical thread 412 on this implementation may have a constant pitch throughout its length. The pitch may be within the range of from about 10 to about 20 threads per inch, or about 5 to about 10 threads per inch depending upon desired performance. Alternatively, the thread may have multiple pitches designed to engage, transport and grasp thrombus within the catheter lumen. A distal pitch may be less than a proximal pitch. The pitch may vary continuously along the length of the thread, or may step from a first, constant pitch in a proximal zone to a second, different pitch in a distal zone of the thread. The thread 412 may comprise a continuous single helical flange, or may have a plurality of discontinuities to produce a plurality of teeth or serrations, arranged helically around the core wire.

The side elevational profile or envelope scribed by the distal tip as it rotates may have a linear or nonlinear taper on one or both ends (e.g., football shaped) which provide varying diameter and thus clearance along its length from the generally cylindrical ID of the catheter lumen.

The maximum OD of the thread 412 is preferably smaller than the diameter of a sliding fit within the catheter lumen, and may generally be at least about 0.015 inches or 0.010 inches smaller than the catheter lumen ID. In some implementations, the Max OD of the tip may be significantly less than the inside diameter of the catheter lumen to allow more space for the thrombus, but still create significant grasping force via engagement of the helical threads with the thrombus. In one implementation, the maximum helical thread diameter is about 0.110 inches and the catheter lumen ID is about 0.275 inches (24 F) (a 0.165 inch gap between the helical threads and catheter wall.

In certain applications, the Max OD of the tip is no more than about 35% or no more than about 40% or no more than about 60% of the ID of the catheter, to leave a substantial tip bypass flow path. Since this implementation does not have any centering structures for the tip 410 or shaft 402, the tip will normally be pushed to one side of the aspiration lumen. When a clot becomes lodged between the tip and the opposing wall of the catheter, manual rotation of the tip can engage the clot like a worm gear and either grasp the clot (e.g., by pinning it against the opposing catheter sidewall) for retraction or facilitate freeing the blockage and aid in ingestion of the clot into the catheter.

The profile of the tip 410 viewed along the axis of rotation may be circular, or may vary to create a non circular pattern around the axis of rotation. The tip as seen in an end elevational view thus exhibits a major diameter and a minor diameter. The minor diameter may be no more than about 95% or 90% or 80% or 70% of the major diameter, depending upon desired performance.

Referring to FIGS. 9A and 9B, the illustrated tip 410 includes a distal advance segment 418 extending between an atraumatic distal tip at 420 and a transition to the distal end 416 of the thread 412. Helical thread 412 extends proximally from the transition to a proximal end 414 of the helical thread 412. A trailing segment 422 extends between the proximal end 414 of the thread and the proximal end 424 of the tip.

The axial length of the advance segment 418 may be at least about 1 cm or 2 cm and in some implementations is within the range of from about 2 cm to about 4 cm. The axial length of the helical thread 412 along the longitudinal axis is typically within the range of from about 1 cm to about 5 cm and in certain implementations between about 2 cm and 3 cm.

The outside diameter of the advance segment 418 at distal tip 420 is generally less than about 0.024 inches, or less than about 0.020 inches and, in one implementation, is about 0.018 inches. The maximum outside diameter of the advance segment 418 and helical thread 412 may be within the range from about 0.020 to about 0.045 inches, and, in one implementation, is less than about 0.040 inches, such as about 0.035 inches. The advance segment, helical thread and trailing segment of the tip 410 may be molded over the flexible shaft 402 using any of a variety of polymers known in the catheter arts.

Referring to FIG. 9B, a first radiopaque marker 430 may be carried on the flexible shaft 402 beneath the advance segment 418. A second radiopaque marker 432 may be carried on the flexible shaft 402 within the trailing segment 422. Each radiopaque marker may comprise a radiopaque tube or a coil of radiopaque wire such as a platinum iridium alloy wire having a diameter about 0.002 inches, and wrapped around the flexible shaft 402 and soldered to the flexible shaft 402 to produce an RO coil having an outside coil diameter of less than about 0.020 inches, such as about 0.012 inches. The radiopaque markers may also function as an axial interference fit between the flexible shaft 402 and the molded advance segment 418 and trailing segment 422 to resist core wire pull out from the tip 410.

In one implementation, the maximum OD of the thread 412 exceeds the maximum OD of the advance segment 418 by at least about 15% or 25% or 30% or more of the OD of the advance segment 418, to facilitate crossing the clot with the advance segment 418 and engaging the clot with the thread 412. The thread pitch may be within the range of from about 0.75 to about 0.30, or within the range of from about 0.10 and about 0.20, such as about 0.14 inches.

Preferably, the maximum OD of the tip 410 is less than about 60% or less than about 40% of the aspiration catheter ID at the distal end of the catheter, and may be within the range of from about 35% to about 55% of the catheter ID. In certain implementations, the maximum OD of the tip 410 may be within the range of from about 0.044 inches to about 0.041 inches within a catheter having a distal end ID within the range from about 0.068 inches to about 0.073 inches.

Depending upon the clinical application, it may be desirable to control the extent to which, if any, the distal tip 410 can extend beyond the distal end of the catheter. For example, distal extension of the distal end of the helical tip beyond the distal end of the catheter may be limited in some implementations to no more than about 5 mm or 3 mm or 1.5 mm or 1.0 mm or less. In other clinical environments the distal tip 420 may be permitted to extend at least about 2 cm or 3 cm and preferably as much as 4 to 8 cm beyond the catheter, but generally will be limited to extend no more than a preset distance such as 12 cm or 8 cm or 5 cm beyond the catheter depending upon desired performance. In one implementation, distal advance of the tip 410 is limited so that the distal end is within 2 cm or within 1 cm or no more than 0.5 cm in either the distal or proximal direction from the distal end of the aspiration catheter.

Distal advance of the tip 420 may be limited by providing mechanical interference at the desired distal limit of travel. In one implementation, a distal stop surface 440 on the handle 408 provides an interference engagement with a complementary proximal surface carried by the aspiration catheter through which the thrombus engagement tool 400 is advanced. Alternatively, a distal engagement surface can be carried anywhere along the length of the thrombus engagement tool 400, for sliding engagement with a complementary proximally facing stop surface carried by the catheter. Additional details may be found in U.S. patent application Ser. No. 17/036,258 filed Sep. 29, 2020 and entitled Embolic Retrieval Catheter, which is hereby expressly incorporated in its entirety herein by reference.

The limit on distal advance of the helical tip may include a first configuration in which distal advance is limited to a first position proximate the distal end of the evacuation catheter to prevent injury to the vascular wall. Upon a user initiated adjustment, the helical tip may be advanced to a second position farther out of the distal end of the catheter such as for inspection and cleaning purposes. This adjustment of the limiting mechanism may be locked out following cleaning or inspection, to limit distal travel to the first position to prevent an undesired degree of exposure of the helical tip element when the system is within the patient's vasculature. Any of a variety of movable interference levers of pins may be engaged to limit travel to the first position, or disengaged to allow travel to the second position.

Referring to FIGS. 10A and 10B, a tip 410 includes a tubular sidewall 440 defining a hub having a connector such as a cavity 442 for coaxially receiving the distal end of a support shaft such as a braid reinforced polyamide tube. The inside diameter of the cavity 442 steps down at a distal end of the hub at a step 444 to a smaller diameter lumen 446 in communication with a distal opening 448. This provides a continuous lumen throughout the length of the micro lumen shaft and tip 410 so that the thrombus engagement tool can be introduced over the wire.

In general, the pitch of thread 412 may be within the range of from about 0.07 to about 0.11, and in one embodiment, is about 0.09. The width of the thread 412 measured along an axis that is perpendicular to a face of the thread may be within the range of from about 0.009 to about 0.04, and, in one embodiment, is about 0.02. The greatest major diameter of the thread 412 may be at least about 10%, or at least about 15%, or at least about 20% greater than the diameter of the proximal hub end of the tip 410 surrounding the cavity 442. In one implementation, the outside diameter of the proximal hub is about 0.090 inches and the outside diameter of the thread 412 is about 0.110 inches. The actual length of the tip 410 including the proximal hub may be within the range of from about 0.2 inches to about 0.8 inches and in some implementations within the range of from about 0.4 inches to about 0.6 inches.

The tip 410 may be manufactured in accordance with any of a variety of techniques known in the art, such as machining, etching, additive and/or subtractive processes. In one implementation, the tip 410 is molded from a polymer such as PEBAX, which may be a 55 D hardness. The PEBAX may include a radiopaque agent, such as bismuth sub carbonate, present in the range of from about 50% to about 70% by weight.

Any of the tip dimensions and configurations disclosed herein may be re-combined with any of the other tip dimensions, configurations, drive shafts and associated structures depending upon the desired clinical performance.

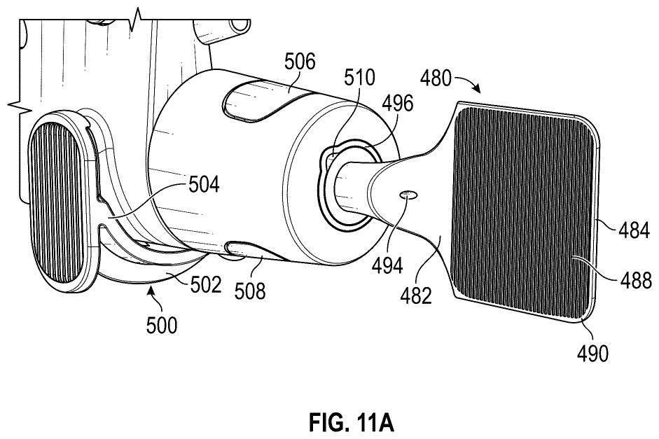

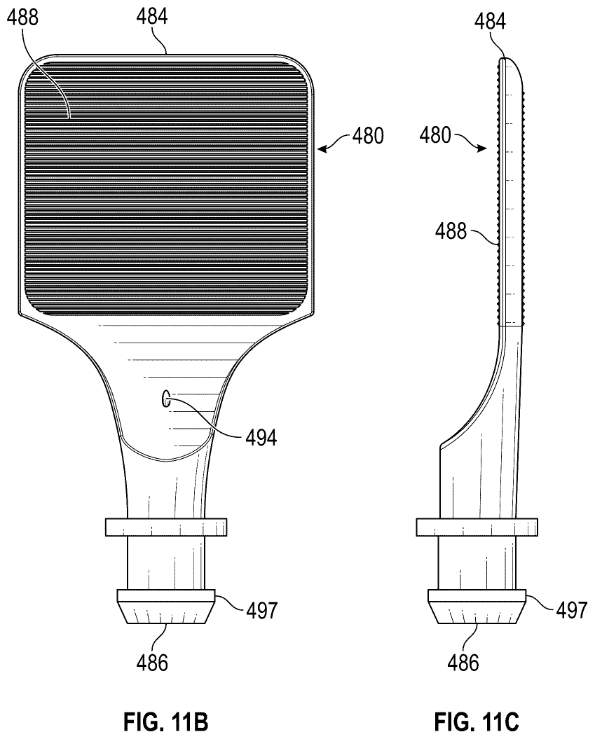

Referring to FIGS. 11A and 20 B, there is illustrated a proximal dilator handle 480. The handle 480 comprises a body 482 having a proximal end 484 a distal end 486 and a longitudinal axis. At least a first proximal gripping surface 488 is carried by the body. In the illustrated implementation, a first gripping surface 488 is provided on at least one side of a paddle shaped grip 490, configured to be held between a thumb and forefinger. A second gripping surface 492 may be provided on an opposing side of the handle. Gripping surfaces may be provided with a friction enhancing surface structures such as a plurality of ridges oriented transverse to the longitudinal axis of the dilator handle 480.

A proximal exit port 494 in communication with the dilator guidewire lumen is oriented along the longitudinal axis of the dilator handle 480, such that a guide wire extending out of the exit port 494 lies along the first gripping surface 488. This allows a clinician to pin the guide wire to the gripping surface 488 using a finger such as a thumb, thereby enabling the dilator and the guide wire to be moved as a unit using one hand.

The dilator may be removably secured to the catheter such as by a retention clip 496 carried by the proximal end of the handle. A release such as a button or deformable interference snap fit may be provided to unlock the dilator handle from the housing, enabling the dilator to be proximally withdrawn from the catheter. In the illustrated implementation, a retention surface such as a proximal surface of a retention ring 497 carried by proximal end 486 of the body 482 provides an interference fit with the retention clip 496. This combines the dilator and handle/catheter into a single system. The paddle may be released from the retention clip by depressing at least a first button 506 and as illustrated also a second button 508 carried on the upper and lower sides of the retention clip housing, and proximally withdrawing the paddle.

This is the same connection and release dock for use with a thrombus engagement tool such as engagement tool 400 discussed in connection with FIGS. 8A and 8B. A distal limit safety feature on the thrombus engagement tool 400 fits into the retention clip 496, ensuring that the distal tip of the tool 400 can not be advanced forward beyond the distal tip of the catheter without both aligning a projection on the tool 400 with the rotational key 502 and intentionally advancing the tool 400 through the retention clip while depressing at least the first button 506 or other unlock control.

Once the distal limit has been released, the tip 410 may be distally advanced no more than about 4 cm and generally about 1 cm to 2 cm beyond the distal end of the catheter. This is intended to be accomplished once the thrombus engagement tool has been withdrawn from the patient, to allow visual inspection of the tip 410.

The engagement tool 400 may also be proximally retracted within the catheter, typically for less than about 3 cm or less than about 2 cm, and may be provided with a spring bias to return to approximate axial alignment between the distal end of the tip 410 and the distal end of the catheter.

A hemostasis clamp 500 may be provided, to hold the hemostasis valve open such as during shipping, or during the advance or withdrawal of devices therethrough. The hemostasis valve is opened by depressing at least a first control button, and in the illustrated implementation first and second control buttons positioned on opposing sides of the handle. The hemostasis clamp comprises a generally U shaped body 502 having a first arm 504 configured to depress a first button, and a second opposing arm (not illustrated) configured to depress a second button on an opposite side of the handle. The hemostasis clamp 500 may be removably retained on the handle by a friction fit, or an interference fit between the handle and the body which can be overcome by plastic deformation as the body is pulled away from the handle to release the hemostasis control buttons.

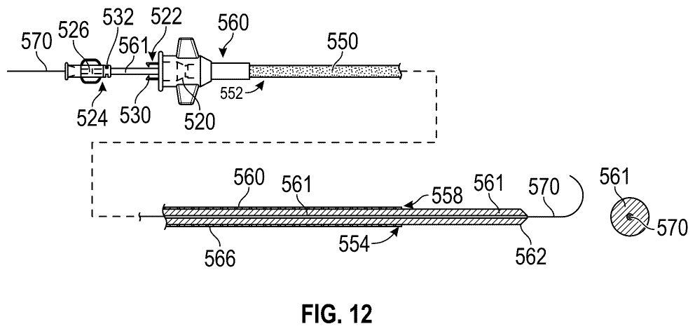

Referring to FIG. 12, an elongate flexible cannulated rail or dilator 561 is shown extending over the guidewire 570 and occupying the space between the guidewire 570 and the large inside diameter of the central lumen 558 of the large diameter catheter 560 to provide support to the catheter and/or an atraumatic tip during delivery.

This catheter-cannulated rail-guidewire assembly is intended to easily track through anatomical challenges more easily than the catheter. The catheter-rail-guidewire assembly then acts as a first stage of the catheter delivery system and enables the large diameter catheter or catheter system to be inserted and independently advanced over this first stage into a blood vessel (e.g. the femoral vein) percutaneously over a guidewire and advanced through potentially tortuous vasculature to the remote target location of interest without requiring advanced skills or causing kinking of the catheter.

The cannulated rail 561 may comprise a soft flexible cylindrical body having a guidewire lumen with a diameter of no more than about 0.040″ and an outside diameter no less than about 0.025″ or about 0.010″ smaller than the inner diameter of the large diameter catheter. Thus the wall thickness of the cannulated rail 561 is typically at least about 0.010″ less than the radius of the large diameter catheter and in some implementations at least about 0.120″ or more, depending upon the size of the annular space between the inside diameter of the catheter and the outside diameter of the guidewire.

The cannulated rail 561 may have an elongated tapered distal tip 562 that may project beyond the distal end 554 of the catheter 560. The thick sidewall of the cannulated rail 561 may comprise one or more flexible polymers, and may have one or more embedded column strength enhancing features such as axially extending wires, metal or polymeric woven or braided sleeve or a metal tube, depending upon the desired pushability and tracking performance along the length of the dilator.

Optionally, the proximal segment of the rail or dilator which is not intended to extend out of the distal end of the catheter may be a structure which is not coaxial with the guidewire, but a control wire which extends alongside the guidewire in the catheter and allows the distal tubular telescoping segment of the rail or dilator to be retracted or extended. (analogous to rapid exchange catheters) without the entire length of the rail structure being over the wire. This allows removal or insertion of the rail or dilator over a shorter guidewire because of the shorter coaxial segment tracking over the guidewire.

Catheter 560 may be provided with a proximal hub 520, having a port for axially movably receiving the rail 561 therethrough. The hub 520 may be provided with an engagement structure such as a first connector 522 for releasably engaging a second complementary connector 524 on a hub 526 on the proximal end of the rail 561. First connector 522 may comprise an interference structure such as at least one radially moveable projection 530, for releasably engaging a complementary engagement structure such as a recess 532 (e.g., an annular ridge or groove) on the hub 526. Distal advance of the rail 561 into the catheter 560 causes the projection 530 to snap fit into the recess 532, axially locking the catheter 560 and rail 561 together so that they may be manipulated as a unit.

The dilator is inserted through the hemostasis valve in the hub 520 of a large bore (e.g., 24 F) catheter 560 and advanced through the catheter until the retention clip on the dilator hub 526 or catheter hub 520 snaps into the complementary recess on the other hub. In this engaged configuration, an advance segment along the flexible distal end of the 24 F rail dilator 561 will extend at least about 5 cm or 10 cm, and in some implementations at least about 15 cm or 20 cm beyond the distal end 554 of the 24 F catheter 560. The rail dilator and 24 F catheter system are thereafter distally advanced over a previously placed guidewire and into the introducer sheath.

The dilator and catheter combination differentiate over prior systems both because of the flexibility of a distal zone of the dilator and greater length of the dilator than the corresponding catheter. Typically, a dilator is a uniform stiffness and length-matched to its catheter, with only a short atraumatic tip of the dilator extending beyond the distal end of the catheter. The dilator has a supportive proximal end and a flexible distal end, with a total dilator length much longer than the catheter 60 to enable, as an example, the following procedure.

In use, a guidewire 570 such as an 0.035″ guidewire is advanced under fluoroscopy using conventional techniques into a selected vessel. The cannulated rail 561, optionally with the catheter 560 mounted thereon, is loaded over the proximal end of the guidewire 570 and advanced distally over the wire until the distal end of the rail is in position at the target site.

The 24 F catheter 560 is thereafter unlocked from the rail 561 and advanced over the rail 561 to the desired site, supported by the rail 561 and guidewire 570 combination. Because the uncovered advance section of the rail has already traversed the challenging tortuosity through the heart, the catheter 561 now just slides over the advance section of the rail for easy passage to the final target location. The supportive proximal zone and flexible distal advance section of the rail enables ease of delivery through the most challenging anatomy in, for example, a PE procedure going from the vena cava through the tricuspid and pulmonary valves of the heart into the central pulmonary artery without concern about damaging the tissue (atraumatic, flexible tip) or damaging the dilator (high kink resistance due to flexible, high wall thickness “solid” dilator construction.

The cannulated rail 561, or the cannulated rail 561 and the guidewire 570 combination, may thereafter be proximally withdrawn, leaving the large bore catheter 560 in position to direct a procedure catheter such as any of the aspiration catheters disclosed elsewhere herein to the target site.

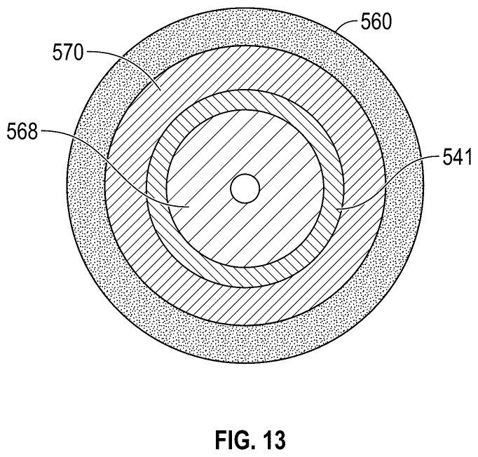

Referring to FIG. 13, the large diameter (LD) catheter 560 may in some situations have a smaller diameter (SD) catheter though its central lumen for the purposes of introducing an additional functionality (e.g., clot grabber catheter 562, imaging catheter 10, or mechanical thrombectomy tool 66) and/or telescoping the SD catheter to more distal locations in the anatomy. In order to enable delivery of the LD catheter 560 and SD catheter as a single system, the SD catheter may have a core dilator 568 for support, and the gap between the outer diameter of the SD catheter and inner diameter of the LD catheter 560 may be maintained or supported by a second, tubular dilator 571. The tubular dilator 571 may have a shaped distal tip 572 for a smooth tapered transition from the SD catheter 541 to the LD catheter 540. The distal end 534 of the core dilator may be provided with a complementary taper to the distal taper of the thin wall SD dilator or may end at the distal end of the LD catheter.

The core dilator 568 inside the SD catheter 541 and tubular dilator 570 between the two catheters may have an interlocking feature to create a single (SD+LD) catheter+ (core+tubular) dilator system. For example, complementary connectors may be provided on hubs on the proximal ends of the system components.

The single (SD+LD) catheter+ (core+tubular) dilator system may be pre-assembled and detachably interlocked at the proximal hub. Additional tubular dilators having a series of outside diameters and wall thicknesses may be provided such that the SD catheter may be used in combination with different diameter LD catheters. A LD catheter may be used with different SD catheters by providing tubular dilators having the same OD but a series of different inside diameters. The core+tubular dilators may simply be pulled proximally to withdraw both dilators as a single system, or the tubular dilator may be configured with a tab or handle at the proximal end and a slit, scoring, perforation or other mechanism so as to split, peel, or tear it along the longitudinal axis during withdrawal to allow the tubular dilator to peel from the SD catheter as it slides proximally out of the space between the LD and SD catheters.

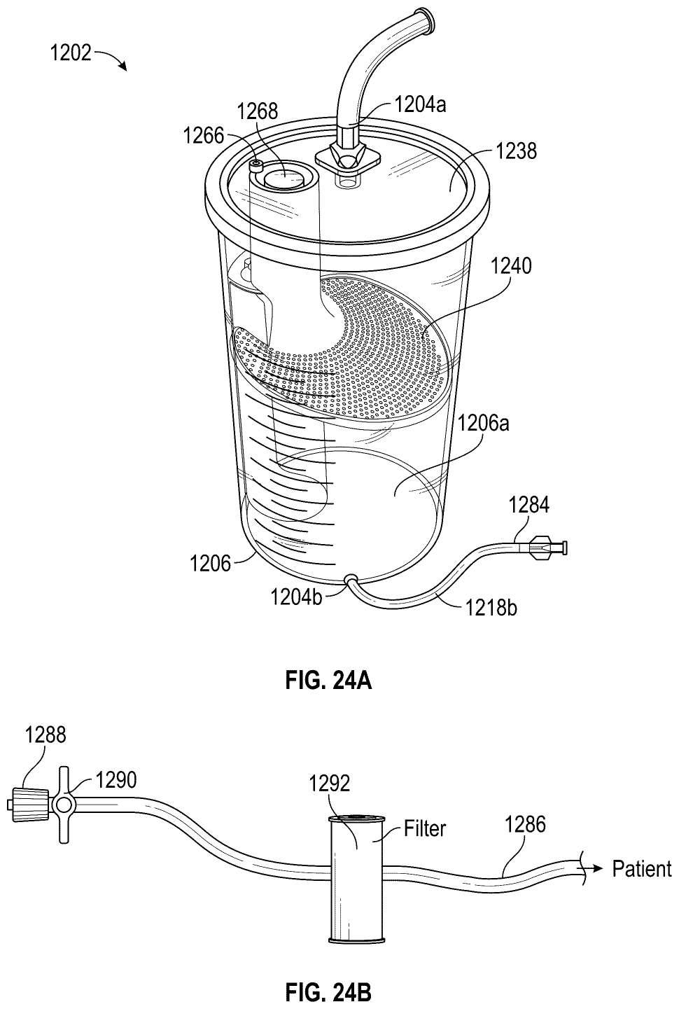

FIG. 24A shows an embodiment of a canister 1202 with a base 1206. While FIG. 24A shows a canister 1202 with a cylindrical shape, it can be appreciated canister 1202 can be formed into other shapes as desired. Canister 1202 can have an open or partially open upper end 1236. The open upper end 1236 can be sealed with a lid 1238 and gasket 1244. In one embodiment, a connector 1246 is attached to canister 1202 and is secured to the lid 1238. In another embodiment, the open upper end 1236 is sealed by the lid 1238 using clasps. In another embodiment, the gasket 1244 is an O-ring. It can be appreciated that a person in the art could use a variety of methods to seal the open upper end 1236 with the lid 1238. Canister 1202 and lid 1238 are typically made of clear plastic.

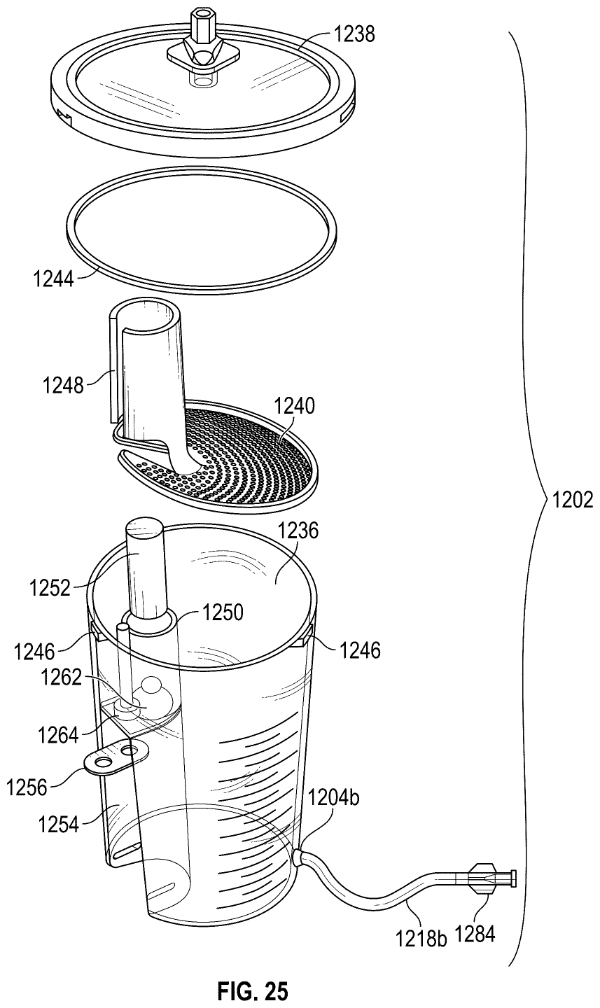

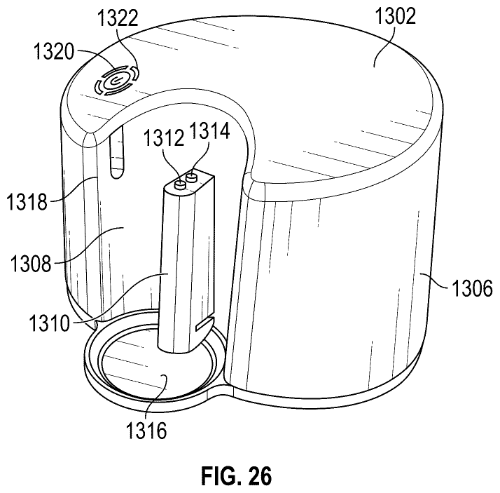

Canister 1202 of FIG. 24A can be used with a vacuum console 1302 of FIG. 26. Canister 1202 can contain at least one groove 1254 in at least one side of the main body and base 1206. Groove 1254 can be shaped such that it can slide onto a complementary post 1310 and the canister 1202 can slide onto a complementary recess 1308 of the enclosure 1306 of the vacuum console 1302. The groove 1254 can have ports or connectors that are configured to be connected to ports or connectors on the post 1310 when the canister 1202 is in recess 1308. The canister 1202 can have a canister post 1250 with a filter body 1252 in the interior of the canister post 1250 which can be configured to prevent extracted material from contaminating the interior of an enclosure 1306 of FIG. 26. FIG. 25 shows an embodiment wherein the groove 1254 has a gasket 1256 to assist in the formation of a seal between the ports on the groove 1254 and the connectors on the post 1310.

The main body of the canister 1202 can have a tube or lumen extending upwardly from the vacuum port 1262 to an aperture 1268 located near the top of the interior of the main body. The aperture 1268 can be configured such that when the lid 1238 is placed over the open upper end 1236, the underside of the lid 1238 will not block flow via aperture 1268. The main body of the canister 1202 can have a tube or lumen extending upwardly from a sensing port 1264 to an aperture 1266 located near the top of the interior of the main body of the canister 1202. The aperture 1266 can be configured such that when the lid 1238 is placed over the open upper end 1236, the underside of the lid 1238 will not block flow via aperture 1266.

Referring to FIG. 24A, the canister 1202 is shown with a first filter 1240. The first filter 1240 can trap solid matter (e.g., thrombus, other particulates, etc.) while allowing fluids (e.g., blood) to flow through the filter 1240. The clot and blood are drawn into canister 1202 by the vacuum console 1302. Clot and blood fall down from inlet 1204a into the canister 1202. Larger clot material collects on the upper surface of the first filter 1240 while blood and smaller clot material passes through the first filter 1240 and collects in a cavity 1206a of the base 1206. The first filter 1240 is made of material capable of separating clot and blood. The first filter 1240 can be configured to be suspended in the interior of the canister 1202, typically at a location between the open upper end 1236 and the base 1206 of the canister 1202. In some cases, the entire perimeter of the first filter 1240 can be in contact with an interior sidewall of the canister 1202, such that blood passes through the first filter 1240 and collects in the cavity 1206a of the base 1206.

The first filter 1240 can be a coarse clot catcher filter, for example with a pore size of about 200 microns. In some cases, the first filter 1240 can include a pore size from about 50 microns and about 400 microns, from about 100 microns to about 350 microns, from about 150 microns to about 300 microns, from about 180 microns to about 270 microns and/or from about 190 microns to about 260 microns. In some cases, the first filter 1240 can include a pore size of about 2000 microns or less. For example, the first filter 1240 can include a pore size from about 500 microns and about 2000 microns, from about 600 microns to about 1500 microns, from about 700 microns to about 1400 microns, from about 800 microns to about 1300 microns, from about 800 microns to about 1200 microns, and/or from about 900 microns to about 1100 microns.

In one embodiment, the first filter 1240 can be made of at least one layer of filter material. In another embodiment, the first filter 1240 can be a composite of different filter materials. In another embodiment, the first filter 1240 can be a composite of filters with different sized pores. In another embodiment, the first filter 1240 can have multiple levels of filters with different sized pores. The first filter 1240 can be shaped or oriented as desired. In another embodiment, a filter can be configured outside of the canister 1202 and upstream of the first filter 1240.

Still referring to FIG. 24A, the base 1206 can have an outlet 1204b connecting the inside and outside of the canister 1202. In another embodiment, the outlet 1204b may instead be located on the wall of the canister 1202. The outlet 1204b can be fluidically coupled to tubing 1218b, In certain embodiments, tubing 1218b can be aspiration tubing. In one embodiment, the outlet 1204b may comprise a valve capable of opening and closing the outlet 1204b. Filtered blood collected in the interior of the base 1206 can pass through the outlet 1204b and through tubing 1218b. The tubing 1218b may comprise an outflow connector 1284. The outflow connector 1284 can be coupled with an outflow port 1288 as shown in FIG. 24B. In another embodiment, the tubing 1218b can include a venous line connected to a patient on one end and to the outlet 1204b on the other end. In some embodiments, a syringe can be fluidically coupled to outlet 1204b and filtered blood drawn from canister 1202. The venous line can beneficially facilitate fluid reinfusion into a patient by drawing fluid collected inside the canister 1202 to the patient via the venous line. Blood filtered by the first filter 1240 and downstream filter 1292 can pass through a reintroduction tubing 1286 and can be introduced into the vasculature of the patient.

Although reference is made to a syringe that can be coupled to the outlet 1204b to withdraw filtered blood from the canister 1202, other devices and methods can be used to withdraw filtered blood from the canister 1202 and/or reintroduce the filtered blood to a patient. For instance, the system can include a pump and/or any pressure source configured to provide force to withdraw blood from the canister 1202 and reintroduce the filtered blood to a patient can be in communication with the canister 1202. The pump and/or device can be in fluid communication with the canister 1202, the outlet 1204b, and/or the tubing 1218b.

Referring now to FIG. 24B, the outflow port 1288 can be configured to open and close with an outflow valve 1290. The outflow valve 1290 may be a user-actuatable valve of any kind known in the art, as desired for the procedure. When the outflow valve 1290 is configured to the open position, blood filtered by the first filter 1240 can pass through the outflow port 1288 and through a downstream filter 1292. The downstream filter 1292 can have pores sized such that blood passing through the downstream filter 1292 is sufficiently filtered to be reintroduced into the vasculature, for example around 40 microns or smaller, around 30-40 microns, or around 20-30 microns. The downstream filter 1292 can be any type of filter known in the art to filter blood suitable for reintroduction into the vasculature.

In some examples, the tubing 1218b can include a venous line connected to a patient on one end and to the outlet 1204b on the other end. The venous line can beneficially facilitate fluid reinfusion into a patient by drawing fluid collected inside the canister 1202 to the patient via the venous line. Blood filtered by the first filter 1240 and/or a second filter 1242 and the downstream filter 1292 passes through a reintroduction tube and can be introduced into the vasculature of the patient. A person skilled in the art would appreciate that other devices may be placed along reintroduction tubing 1286 and/or coupled to the outlet 1204b as desired. For example, filtered blood can be withdrawn from the canister 1202 by coupling a syringe to the outlet 1204b and using the syringe to withdraw blood the canister for reintroduction into a patient. In some cases, a blood reintroduction line can be connected to a patient on one end and to the outlet 1204b on the other end. The blood reintroduction line can be in fluid communication with a pump and/or any device configured to provide force to drive the filtered blood from the canister 1202 to a patient via the blood reintroduction line.

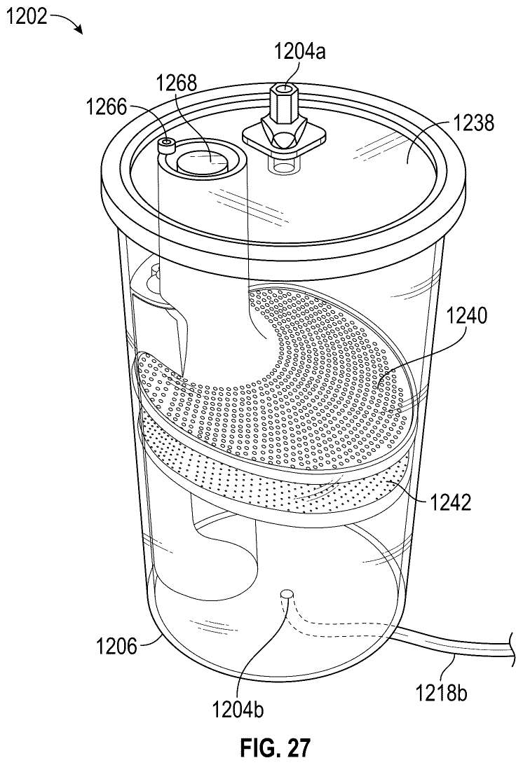

Although reference is made to the canister 1202 shown in FIG. 27 including a first filter 1240 and a second filter 1242, the canister 1202 can include more than or less than two filters. For example, the canister 1202 can include a single filter (e.g., only the first filter 1240; only the second filter 1242) and/or three, four, five, etc. filters.

In one embodiment, a pressure source can be fluidically coupled to reintroduction tubing 1286. The pressure source can be the same or different than the pressure source used to generate pressure for the aspiration of clot material. In some embodiments, the pressure source can draw blood and clot through the first filter 1240, the second filter 1242, and/or the downstream filter 1292. In some embodiments, the pressure source can be an electric pump. In one embodiment, a user-actuatable valve is coupled to the reintroduction tubing 1286 such that blood reintroduction to the patient can be stopped and started as desired. In another embodiment, a blood reservoir can be coupled to the reintroduction tubing 1286 such that filtered blood can be collected and reintroduced to the patient as desired.

FIG. 26 shows an embodiment of the vacuum console 1302, shown without canister 1202. Vacuum console 1302 comprises an enclosure 1306 which can house a pressure source. Vacuum console 1302 can have a recess 1308, which is shaped to receive canister 1202. A post 1310 can be formed within recess 1308 and can be shaped to receive canister 1202. A bottom plate 1316 can extend laterally from the base of enclosure 1306 and can be at least partially shaped to complement recess 1308. Bottom plate 1316 can provide support for canister 1202 when canister 1202 is received within recess 1308. A vacuum connector 1312 and a sensing connector 1314 can be formed on post 1310. Vacuum connector 1312 is configured to align with a vacuum port 1262 on the canister 1202, for example as shown in FIG. 25. Sensing connector 1314 is configured to align with a sensing port 1264 of the canister 1202, for example as shown in FIG. 25. In one embodiment, sensing connector 1314 can be a pressure sensing connector. Vacuum connector 1312 and sensing connector 1314 are configured to align with the vacuum port 1262 and sensing port 1264 when the canister 1202 is received within recess 1308. FIG. 26 shows an embodiment wherein the post 1310 has sensing connector 1314 and vacuum connector 1312 configured to be connected to sensing port 1264 and vacuum port 1262, respectively, in the groove 1254. In one embodiment, the groove 1254 has a blood reintroduction port that is configured to be connected to a blood reintroduction connector on the post 1310 when the canister 1202 is in recess 1308. The port or connectors on the post 1310 can be sealed to the complementary ports or connectors on the groove 1254.

Continuing to refer to FIG. 26, a user interface can be located on at least one wall or top of vacuum console 1302. In an exemplary embodiment, the user interface can be comprised of a switch 1320, a pressure indicator 1322, and a power indicator 1318. In one embodiment, switch 1320 can be user-actuated to power vacuum console 1302 on or off. When vacuum console 1302 is powered on, power indicator 1318 is illuminated. When vacuum console 1302 is generating pressure, pressure indicator 1322 is illuminated. In one embodiment, pressure indicator 1322 can comprise at least one light that can illuminate in sequence to indicate the vacuum level. FIG. 26 shows an embodiment in which pressure indicator 1322 comprises four segments which can be sequentially illuminated to show the vacuum level within the canister as a percentage of ambient pressure. In another embodiment, pressure indicator 1322 can comprise at least one light that can change colors to indicate the vacuum level. User interface on vacuum console 1302 can comprise additional indicators as desired, for example a light indicating a secure connection when canister 1202 is received in recess 1308.

Referring to FIG. 27, the canister 1202 is shown with first filter 1240 and a second filter 1242. In an embodiment, first filter 1240 is a coarse clot filter and second filter 1242 is a finer filter than first filter 1240. First filter 1240 and second filter 1242 are sized to allow for filtering blood such that the blood passing through both filters can be reintroduced into the body. First filter 1240 and second filter 1242 can touch all sides of the wall of the interior of canister 1202, such that blood must pass through first filter 1240 and second filter 1242 to collect in the cavity 1206a of the base 1206. In some cases, first filter 1240 creates a seal with the interior of canister 1202 while the second filter 1242 does not create a seal. In some cases, first filter 1240 does not create a seal with the interior of canister 1202 while the second filter 1242 does create a seal with the interior of canister 1202. In some cases, both first filter 1240 and second filter 1242 create seals with the interior of canister 1202.

The wall of the first filter 1240 can be a coarse clot catcher filter, for example with a pore size of about 200 microns, or between about 50 microns and about 400 microns, between about 100 microns to about 350 microns, between about 150 microns to about 300 microns, and/or between about 180 microns to about 270 microns.

The second filter 1242 can have pores sized such that blood passing through the second filter 1242 is sufficiently filtered to be reintroduced into the vasculature (e.g., at least substantially free of clots), for example about 40 microns, or between about 20 microns and about 80 microns, between about 30 microns and about 70 microns, between about 40 microns and about 60 microns, and/or between about 45 microns to about 55 microns. The second filter 1242 can be any type of filter known in the art to filter blood suitable for reintroduction into the vasculature. In some cases, the second filter 1242 can include a pore size of about 200 microns or less. For example, the second filter 1242 can include a pore size from about 10 microns to about 200 microns, from about 30 microns to about 180 microns, from about 50 microns to about 150 microns, from about 70 microns to about 130 microns, from about 80 microns to about 120 microns, from about 90 microns to about 110 microns, and/or from about 95 microns to about 105 microns.

In some embodiments, first filter 1240 and/or second filter 1242 can be made of at least one layer of filter material. In another embodiment, first filter 1240 and/or second filter 1242 can be a composite of different filter materials. In another embodiment, first filter 1240 and/or second filter 1242 can be a composite of filters with different sized pores. In another embodiment, first filter 1240 and/or second filter 1242 can have multiple levels of filters with different sized pores. First filter 1240 and second filter 1242 can be shaped or oriented as desired.

Any clots and/or blood can be drawn into the interior of the main body of canister 1202 by the vacuum console 1302. The clots and/or blood can fall down from an inlet 1204a into canister 1202. In some cases, a catheter, also referred to herein as an aspiration catheter, can be fluidically connected to the inlet 1204a. In some instances, the catheter can be connected to the vacuum console 1302. The vacuum console 1302 can be configured to drive the blood through the canister 1202. In some instances, the pressure source can be a different pressure source than the vacuum console 1302. In some instances, a syringe can be fluidically connected to the inlet 1204a. In some instances, the syringe can contain blood. In some instances, the syringe can contain unfiltered blood.

Larger clot material can be collected on the upper surface of the first filter 1240 while blood and smaller clot material can pass through the first filter 1240. The smaller clot material can be collected on the upper surface of second filter 1242 while blood passes through second filter 1242 and is collected in the cavity 1206a of the base 1206. The first filter 1240 and the second filter 1242 can be suspended in the interior of the canister 1202, typically at a location between the open upper end 1236 and the canister base 1206. The second filter 1242 can be positioned at a location below the location of first filter 1240. The entire perimeter of the first filter 1240 and/or the second filter 1242 can be in contact with an interior side wall of the canister 1202. This can beneficially result in blood flowing through the first filter 1240 and/or second filter 1242 before being collected in the interior of the canister base 1206.

Still referring to FIG. 27, the base 1206 of the canister 1202 can have an outlet 1204b. The outlet 1204 can be in fluid communication with the inside and outside of the canister 1202. In another embodiment, outlet 1204b may instead be located on the wall of the canister 1202. The outlet 1204b can be fluidically coupled to the tubing 1218b. In one embodiment, the outlet 1204b can include a valve capable of opening and closing the outlet 1204b. Filtered blood collected in the interior of the base 1206 can pass through the outlet 1204b and through the tubing 1218b. The tubing 1218b may be connected to additional components. For example, the tubing 1218b can be connected to the outflow connector 1284, which is described in relation to FIGS. 24A and 25. The outflow connector 1284 can be coupled with an outflow port, such as the outflow port 1288 shown in FIG. 24B. In another embodiment, the tubing 1218b can include a venous line connected to a patient on one end and to the outlet 1204b on the other end. The venous line can beneficially facilitate fluid reinfusion into a patient by drawing fluid collected inside the canister 1202 to the patient via the venous line.

In some embodiments, a pressure source can be fluidically coupled to tubing 1218b. The pressure source can be configured to draw fluid collected inside the canister 1202 to the patient via the venous line. Blood filtered by the first filter 1240 and second filter 1242 can pass through reintroduction tubing and can be introduced into the vasculature of the patient. In one case the reintroduction tubing can be reintroduction tubing 1286 which is described in relation to FIGS. 24 and 25. In some embodiments, the pore size of first filter 1240 and second filter 1242 may determine whether the blood should be further filtered by, for example, downstream filter 1292 prior to reintroduction into the vasculature of the patient.

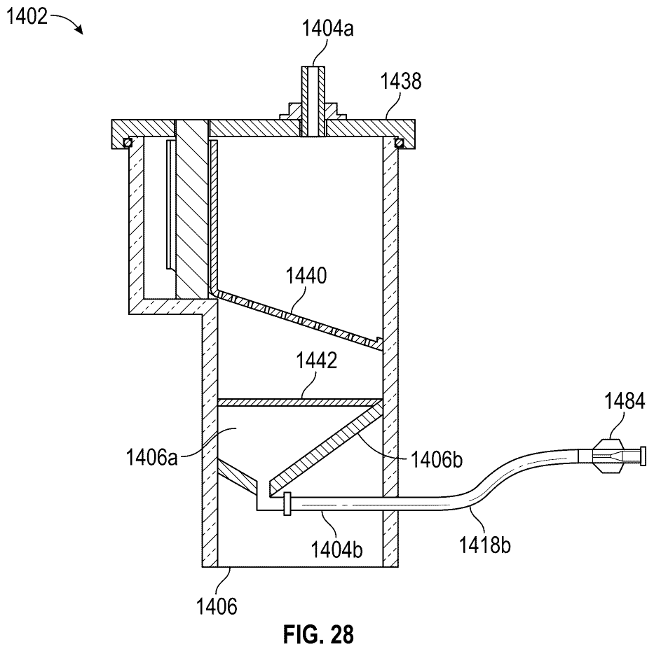

FIG. 28 shows another example of a canister 1402 for use with a blood reintroduction system. The canister 1402 can comprise a lid 1438, an inlet 1404a, an outlet 1404b, and a base 1406. The inlet 1404a and the outlet 1404b can be connected to other medical devices via tubing. In some cases, a catheter can be fluidically connected to the inlet 1404a. In some instances, the catheter is connected to a pressure source configured to drive blood through the canister 1402. In some instances, a syringe can be fluidically connected to inlet 1404a. In some instances, the syringe can contain blood. In some instances, the syringe can contain unfiltered blood. The contents of the syringe can be deposited into the canister 1402 via the inlet 1404a. In some cases, a syringe can be fluidically coupled to the outlet 1404b to withdraw the filtered blood from the canister 1402. In some cases, aspiration tubing can be connected to the inlet 1404a. The aspiration tubing can place the canister 1402 and an aspiration system, such as aspiration system 100 (as described in relation to FIG. 1) in fluid communication with each other. Blood drawn into the canister 1402 via the aspiration tubing can collect in a cavity 1406a. Floor 1406b can be sloped such that it directs blood in a desired direction. In some embodiments floor 1406b can be a funnel shape. In some embodiments floor 1406b with a funnel shape can direct blood towards the middle of the funnel. Floor 1406b can be the same or different as base 1406. In some embodiments, floor 1406b can be elevated above base 1406 such that no part of floor 1406b touches base 1406. Floor 1406b can have all, some, or no contact with the bottom of the canister 1402.

Canister 1402 can have an outlet 1404b. The outlet 1404b can be fluidically coupled to tubing 1418b, In certain embodiments, tubing 1418b can be aspiration tubing. Canister 1402 can include one or more filters. The one or more filters can be positioned upstream of the outlet 1404b, along tubing 1418b, and/or along a flow path (e.g., a venous line between the outlet 1404b and the patient) before reinfusion of the fluid to the patient. In some embodiments, a pressure source can be fluidically coupled to tubing 1418b. The pressure source can be configured to draw fluid collected inside the canister 1402 to the patient via the venous line. In the embodiment shown in FIG. 28, canister 1402 has a first filter 1440 and second filter 1442. The first filter 1440 can be positioned above the second filter 1442. At least one of the first filter 1440 and the second filter 1442 can be positioned inside the cavity 1406a. In some cases, a shape and/or dimensions of the cavity 1406a are larger than a shape and/or dimensions of the at least one of first filter 1440 and second filter 1442 to allow the cavity 1406a to receive and secure the filters. The first filter 1440 and second filter 1442 can prevent solid matter, such as thrombus, collected inside the canister 1402 from reaching the outlet 1404b. The first filter 1440 and second filter 1442 can trap solid matter while allowing fluids (e.g., blood), to flow past the filters. In some cases, the outlet 1404b can extend from the lowest portion of the floor 1406b. at an angle.

In some cases, a longitudinal axis of the outlet 1404b can incline from a plane defined by the base 1406 at an angle. In some cases, the angle can be an acute angle. For example, the angle can be between about 10° and about 80°. For example, the angle can be between about 10° and about 40°, between about 20° and about 50°, between about 30° and about 60°, between about 40° and about 70°, or between about 50° and about 80°. In some cases, the angle can be about 45°. In some cases, the angle can be a right angle.

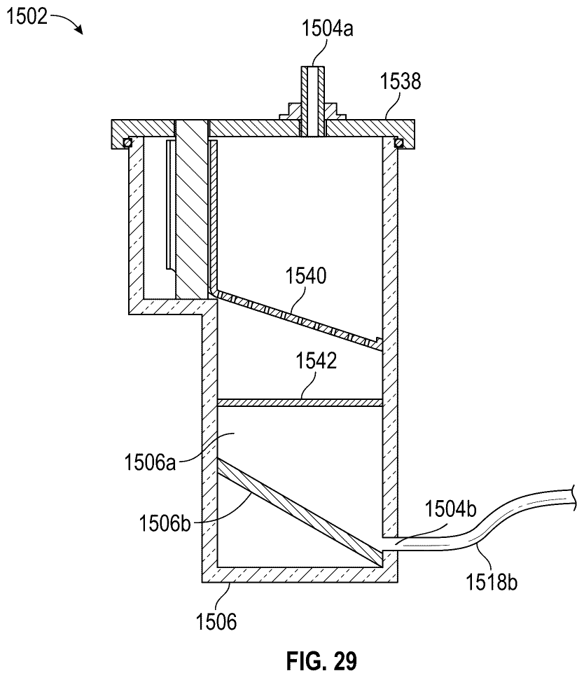

FIG. 29 shows another example of a canister 1502 for use with a blood reintroduction system. Like the canister 1402, the canister 1502 can comprise a lid 1538, an inlet 1504a, an outlet 1504b, and a base 1506. The inlet 1504a and the outlet 1504b can be connected to other medical devices via tubing. In some cases, a catheter can be fluidically connected to the inlet 1504a. In some instances, the catheter is connected to a pressure source configured to drive blood through the canister 1502. In some instances, a syringe can be fluidically connected to inlet 1504a. In some instances, the syringe can contain blood. In some instances, the syringe can contain unfiltered blood. The contents of the syringe (e.g., unfiltered blood) can be deposited into the canister 1502 via the inlet 1504a. In some cases, a syringe can be fluidically coupled to the outlet 1504b to withdraw the filtered blood from the canister 1502. In some cases, aspiration tubing can be connected to the inlet 1504a. The aspiration tubing can place the canister 1502 and an aspiration system, such as aspiration system 100 (as described in relation to FIG. 1) in fluid communication with each other. Blood drawn into the canister 1502 via the aspiration tubing can collect in a cavity 1506a. Floor 1506b can be sloped such that it directs blood in a desired direction. Floor 1506b can be the same or different as base 1506. Floor 1506b can have all, some, or no contact with the bottom of the canister 1502.

Canister 1502 can have an outlet 1504b. The outlet 1504b can be fluidically coupled to tubing 1518b, In certain embodiments, tubing 1518b can be aspiration tubing. Canister 1502 can include one or more filters. The one or more filters can be positioned upstream of the outlet 1504b, along tubing 1518b, and/or along a flow path (e.g., a venous line between the outlet 1504b and the patient) before reinfusion of the fluid to the patient. In some embodiments, a pressure source can be fluidically coupled to tubing 1518b. The pressure source can be configured to draw fluid collected inside the canister 1502 to the patient via the venous line. In the embodiment shown in FIG. 29, canister 1502 has a first filter 1540 and second filter 1542. The first filter 1540 can be positioned above the second filter 1542. At least one of the first filter 1540 and the second filter 1542 can be positioned inside the cavity 1506a. In some cases, a shape and/or dimensions of the cavity 1506a are larger than a shape and/or dimensions of the at least one of first filter 1540 and second filter 1542 to allow the cavity 1506a to receive and secure the filters.

The first filter 1540 and second filter 1542 can prevent solid matter, such as thrombus, collected inside the canister 1502 from reaching the outlet 1504b. The first filter 1540 and second filter 1542 can trap solid matter while allowing fluids (e.g., blood), to flow past the filters. In some cases, the outlet 1504b can extend from the lowest portion of the floor 1506b. at an angle. In some cases, a longitudinal axis of the outlet 1504b can incline from a plane defined by the base 1506 at an angle. In some cases, the angle can be an acute angle. For example, the angle can be between about 10° and about 80°. For example, the angle can be between about 10° and about 40°, between about 20° and about 50°, between about 30° and about 60°, between about 40° and about 70°, or between about 50° and about 80°. In some cases, the angle can be about 45°. In some cases, the angle can be a right angle.

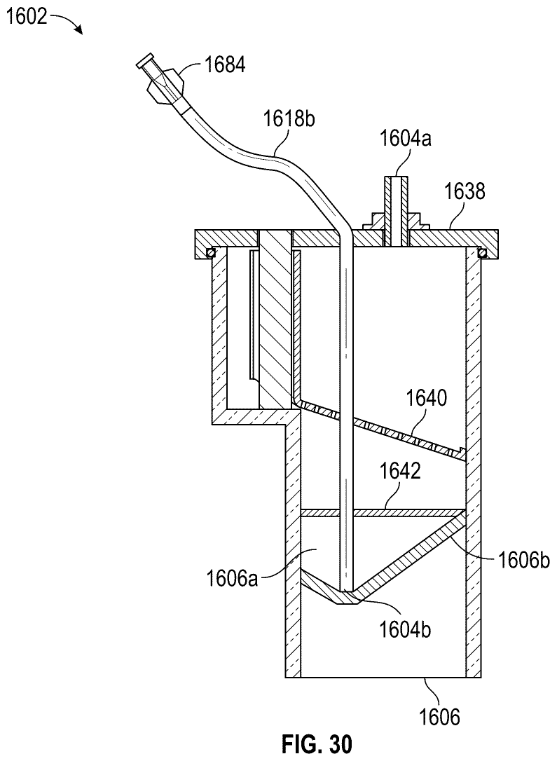

FIG. 30 shows another example of a canister 1602 for use with a blood reintroduction system. Like the canister 1402 and 1502, the canister 1602 can comprise a lid 1638, an inlet 1604a, an outlet 1604b, and a base 1606. The inlet 1604a and the outlet 1604b can be connected to other medical devices via tubing. In some cases, a catheter can be fluidically connected to the inlet 1604a. In some instances, the catheter is connected to a pressure source configured to drive blood to and through the canister 1602. In some instances, a syringe can be fluidically connected to inlet 1604a. In some instances, the syringe can contain blood. In some instances, the syringe can contain unfiltered blood. The contents (e.g., unfiltered blood) of the syringe can be deposited into the canister 1602 via the inlet 1604a for filtering. In some cases, a syringe can be fluidically coupled to outlet 1604b and filtered blood drawn from canister 1602. In some cases, aspiration tubing can be connected to the inlet 1604a. The aspiration tubing can place the canister 1602 and an aspiration system, such as aspiration system 100 (as described in relation to FIG. 1) in fluid communication with each other. Blood drawn into the canister 1602 via the aspiration tubing can collect in a cavity 1606a. Floor 1606b can be sloped such that it directs blood in a desired direction. Floor 1606b can be the same or different as base 1606. Floor 1606b can have all, some, or no contact with the bottom of the canister 1602.

Canister 1602 can have an outlet 1604b. The outlet 1604b can be fluidically coupled to tubing 1618b, In certain embodiments, tubing 1618b can be aspiration tubing. Canister 1602 can include one or more filters. The one or more filters can be positioned upstream of the outlet 1604b, along tubing 1618b, and/or along a flow path (e.g., a venous line between the outlet 1604b and the patient) before reinfusion of the fluid to the patient. In some embodiments, a pressure source can be fluidically coupled to tubing 1618b. The pressure source can be configured to draw fluid collected inside the canister 1602 to the patient via the venous line. In the embodiment shown in FIG. 30, canister 1602 has a first filter 1640 and second filter 1642. The first filter 1640 can be positioned above the second filter 1642. At least one of the first filter 1640 and the second filter 1642 can be positioned inside the cavity 1606a.

In some cases, a shape and/or dimensions of the cavity 1606a are larger than a shape and/or dimensions of the at least one of first filter 1640 and second filter 1642 to allow the cavity 1606a to receive and secure the filters. The first filter 1640 and second filter 1642 can prevent solid matter, such as thrombus, collected inside the canister 1602 from reaching the outlet 1604b. The first filter 1640 and second filter 1642 can trap solid matter while allowing fluids (e.g., blood), to flow past the filters. In some cases, the outlet 1604b can extend from the lowest portion of the floor 1606b. at an angle. In some cases, a longitudinal axis of the outlet 1604b can incline from a plane defined by the base 1606 at an angle. In some cases, the angle can be an acute angle. For example, the angle can be between about 10° and about 80°. For example, the angle can be between about 10° and about 40°, between about 20° and about 50°, between about 30° and about 60°, between about 40° and about 70°, or between about 50° and about 80°. In some cases, the angle can be about 45°. In some cases, the angle can be a right angle. In the embodiment shown in FIG. 30, tubing 1618b forms a right angle with the longitudinal axis from a plane defined by the base 1606. The tubing 1618b extends from floor 1606b, through second filter 1642 and first filter 1640 and out of lid 1638.

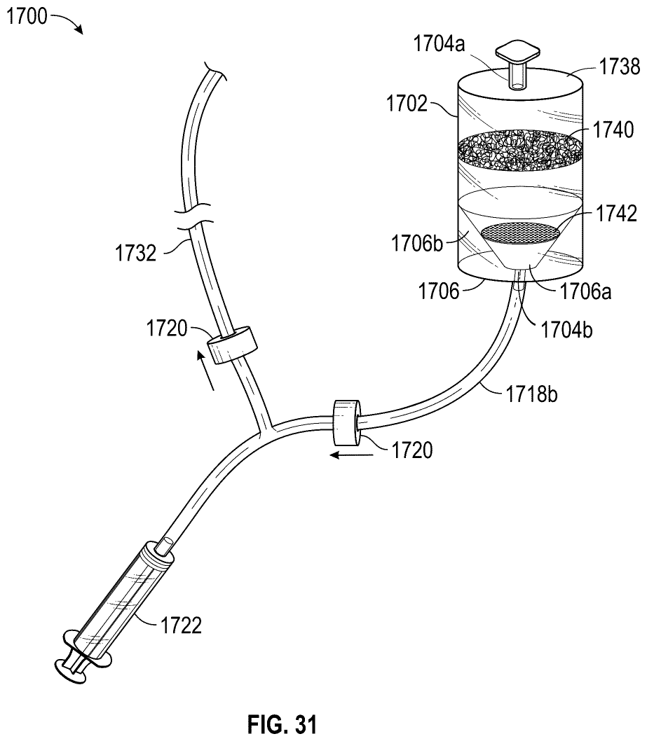

FIG. 31 shows an example of a blood reintroduction system 1700. Blood reintroduction system 1700 shows an example of a canister 1702 for use with a blood reintroduction system. Like the canister 1402, 1502 and 1602, the canister 1702 can comprise a lid 1738, an inlet 1704a, an outlet 1704b, and a base 1706. The inlet 1704a and the outlet 1704b can be connected to other medical devices via tubing. In some cases, a catheter, such as aspiration catheter, can be fluidically connected to the inlet 1704a. The catheter can be connected to a pressure source configured to drive blood from a patient to the canister 1702. In some instances, one or more syringes can be fluidically connected to the inlet 1704a. The syringe can contain blood. In some instances, the syringe can contain unfiltered blood. The blood from the catheter and/or the syringe can be delivered to the canister 1702 via the inlet 1704a for filtering, additional filtering, and/or reintroduction into a patient as further described herein. In some cases, a syringe or any other reciprocating pumping mechanism can be fluidically coupled to the outlet 1704b to draw filtered blood from the canister 1702.

In some cases, aspiration tubing can be connected to the inlet 1704a. The aspiration tubing can place the canister 1702 and an aspiration system, such as aspiration system 100 (as described in relation to FIG. 1) in fluid communication with each other. Blood drawn into the canister 1702 via the aspiration tubing can collect in a cavity 1706a. Floor 1706b can be sloped such that it directs blood in a desired direction. Floor 1706b can be the same or different as base 1706. Floor 1706b can have all, some, or no contact with the bottom of the canister 1702.

Canister 1702 can have an outlet 1704b. The outlet 1704b can be fluidically coupled to tubing 1718b, In certain embodiments, tubing 1718b can be aspiration tubing. Canister 1702 can include one or more filters. The one or more filters can be positioned upstream of the outlet 1704b, along tubing 1718b, and/or along a flow path (e.g., a venous line between the outlet 1704b and the patient) before reinfusion of the fluid to the patient. In the embodiment shown in FIG. 31, canister 1702 has a first filter 1740 and second filter 1742. The first filter 1740 can be positioned above the second filter 1742.

In some cases, second filter 1742 can be positioned downstream of the outlet 1704b. For example, the second filter 1742 can be positioned along the flow path 1732, between the valves 1720, and/or along the tubing 1718b. Although reference is made to the second filter 1742 being positionable at different locations of the blood reintroduction system 1700, the second filter 1742 can be positioned anywhere upstream of the patient. This can beneficially allow the second filter 1742 to capture any remaining clots in the blood to be reintroduced to the patient. In some cases, the blood reintroduction system can include a plurality of filters that can be positioned inside the canister 1702 and/or upstream of the outlet 1704b. For example, the blood reintroduction system 1700 can include a first filter 1740 inside the canister 1702, a second filter 1742 inside the canister 1702, and one or more filters positioned along the tubing 1718b, between the valves 1720, and/or along the flow path 1732.

In some cases, at least one of the first filter 1740 and the second filter 1742 can be positioned inside the cavity 1706a. In some cases, a shape and/or dimensions of the cavity 1706a are larger than a shape and/or dimensions of the at least one of first filter 1740 and second filter 1742 to allow the cavity 1706a to receive and secure the filters. The first filter 1740 and second filter 1742 can prevent solid matter, such as thrombus, collected inside the canister 1702 from reaching the outlet 1704b. The first filter 1740 and second filter 1742 can trap solid matter while allowing fluids (e.g., blood), to flow past the filters. In some cases, the outlet 1704b can extend from the lowest portion of the floor 1706b. at an angle. In some cases, a longitudinal axis of the outlet 1704b can incline from a plane defined by the base 1706 at an angle. In some cases, the angle can be an acute angle. For example, the angle can be between about 10° and about 80°. For example, the angle can be between about 10° and about 40°, between about 20° and about 50°, between about 30° and about 60°, between about 40° and about 70°, or between about 50° and about 80°. In some cases, the angle can be about 45°. In some cases, the angle can be a right angle. In the embodiment shown in FIG. 30, tubing 1718b forms a right angle with the longitudinal axis from a plane defined by the base 1706 and extends from floor 1706b, through base 1706.

Still referring to FIG. 31, the blood reintroduction system 1700 can have a pressure source such as a syringe 1722 and/or a pump. Blood flowing through the outlet 1704b can be drawn by the syringe 1722 through at least one valve 1720. In some cases, the at least one valve 1720 can be positioned along flow path 1732 and/or the tubing 1718b. The tubing 1718b can include one or more valves 1720. The valves 1720 can be one-way valves and/or user actuatable. In some cases, the valves 1720 can include a stopcock valve. The valves 1720 can be different types of valves or the same type of valves. In some cases, the syringe 1722 can be cycled (e.g., by pushing and/or pulling a plunger of the syringe) to flow the blood downstream of the valves 1720. In embodiments where the valves 1720 include one-way valves, the valves 1720 can prevent the blood from upstream when the syringe 1722 is cycled. This can beneficially prevent filtered blood from flowing upstream (e.g., back to the canister 1702) and instead ensure that the filtered blood flows back to the patient for reintroduction.

Opening and/or closing the valve 1720 along the tubing 1718b can beneficially allow users to control aspiration and/or the flow of blood through the tubing 1718b. In some cases, the outlet 1704b can include a valve such as a luer fitting. Fluid 630 extracted from inside the canister 1702 via the outlet 1704b can be reintroduced to a patient. For example, the fluid extracted by the extraction device, such as the syringe 1722, can be injected directly or indirectly to a patient. In some cases, the fluid can be injected into the vasculature of a patient. The fluid can also be injected to a patient from the syringe 1722 to, for example, a venous line connected to a patient. In some cases, the blood reintroduction system 1700 can include a venous line connected to a patient on one end and to the outlet 1704b on the other end, such as flow path 1732. In some cases, flow path 1732 can include tubing. In some cases, flow path 1732 can be tubing 1718b.

To preserve the ability to visually inspect the blood and/or clots in the blood reintroduction system 1700, it may be necessary to clear blood from the canister 1702, the flow path 1732, and/or the tubing 1718b. In some cases, the tubing 1718b and/or the flow path 1732 can comprise an inlet for flushing or priming. In such instances, a syringe can be fluidically connected to the inlet to flush the tubing, for example with saline. In some cases, cleaning the flow path 1732 and/or the tubing 1718 may be desirable between rounds of blood reintroduction. Thus, the reintroduction system 1700 can be flushed and/or cleaned, for example, between predefined periods of time (e.g., every 5, 10, 20, 40, 60, 80, 120, seconds, etc.), after receiving blood from a patient, after blood is reintroduced to the patient, and/or when visual inspection of the interior of the canister 1702 is no longer possible. In some instances, the inlet can comprise a hemostasis valve such that the tubing 1718b and/or the flow path 1732 can be flushed without introducing air into the line.

The venous line can beneficially facilitate fluid reinfusion into a patient by drawing fluid collected inside the canister 1702 to the patient via the venous line. In such cases, it may not be necessary for clinicians to manually extract fluid from the canister 1702 and reinfuse the fluid to the patient. That is, fluid collected inside the canister 1702 can be automatically reinfused by flowing though the venous line into the vasculature of patient or a flow path into a vasculature of patient, such as flow path. Reinfusion may be assisted by a pump in between the outlet 1704b and the patient (not illustrated). In some embodiments, a pressure source can be fluidically coupled to tubing 1718b. The pressure source can be configured to draw fluid collected inside the canister 1702 to the patient via the venous line.

In some cases, the filtered blood can be returned quickly to the patient, for example within a few seconds. In some cases, the filtered blood can be returned (e.g., reintroduced) to the patient within a few minutes. In some cases, the filtered blood can be stored for later reinfusion. In some cases, the blood reintroduction system 1700 can be configured to minimize blood volume downstream of the outlet 1704b, for example to reduce the risk of clotting. In some cases, the diameter of the tubing in the tubing 1718b and/or flow path 1732 can configured to minimize blood volume downstream of outlet 1704b to reduce the risk of clotting. In some cases, the length of the tubing in the tubing 1718b and/or the flow path 1732 can be configured to minimize blood volume downstream of outlet 1704b to reduce the risk of clotting.

In any of the blood reintroduction systems described herein, filtered blood can be returned to the patient via a patient line. For example, filtered blood drawn from any blood reintroduction systems can reintroduced back into a patient via a patient line, such as the patient line 202 of the catheter handle 140, which is described in relation to FIGS. 7A and 7B. As described in relation to FIGS. 7A and 7B, the patient line 202 may be placed in communication with a manifold line 204 by actuating the valve 200 to a predefined position to allow the reintroduction of filtered blood. The filtered blood can be delivered to a catheter handle (e.g., catheter handle 106, 140) via a syringe and/tubing in fluid communication with a manifold (e.g., manifold line 204).

Any of the blood reintroduction systems described herein can be automated to return blood to the patient based on adequate volume of filtered blood available. For example, the blood reintroduction systems described herein can include a sensor configured to detect an amount of filtered blood within the blood reintroduction system (e.g., within a canister). In such cases, the blood reintroduction system to configured to activate a pump (or any other vacuum device) to return the filtered blood to a patient upon, for example, detecting a threshold amount of filtered blood within the canister. The threshold amount can be, for example, more than or less than about 1 cc, 2 cc, 5 cc, 10 cc, 15 cc, 20 cc, 25 cc, 30 cc, 50 cc, 60 cc, 100 cc, 200 cc, 500 cc, etc.

Any of the blood reintroduction systems described herein can include a sensor to detect air within the blood reintroduction system and/or its components. This can beneficially prevent or reduce the introduction of air into the patient. The blood reintroduction systems described herein can also include sensors to detect pressure and/or other aspects to verify, for example, that the filtered blood is flowing into the patient and not against high resistance.

While certain arrangements of the inventions have been described, these arrangements have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, arrangement, or example are to be understood to be applicable to any other aspect, arrangement or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing arrangements. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some arrangements, the actual steps taken in the processes illustrated and/or disclosed may differ from those shown in the figures. Depending on the arrangement, certain of the steps described above may be removed, others may be added. Furthermore, the features and attributes of the specific arrangements disclosed above may be combined in different ways to form additional arrangements, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular arrangement. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain arrangements include, while other arrangements do not include, certain features, elements, and/or steps. Thus, such conditional language is not generally intended to imply that features, elements, and/or steps are in any way required for one or more arrangements or that one or more arrangements necessarily include logic for deciding, with or without user input or prompting, whether these features, elements, and/or steps are included or are to be performed in any particular arrangement.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain arrangements require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain arrangements, the terms “generally parallel” and “substantially parallel” refer to a value, amount, or characteristic that departs from exactly parallel by less than or equal to 15°, 10°, 5°, 3°, 1 degree, or 0.1 degree. The ranges disclosed herein also encompass any and all overlap, sub-ranges, and combinations thereof, and any specific values within those ranges. Language such as “up to,” “at least,” “greater than,” “less than,” “between,” and the like includes the number recited. Numbers and values used herein preceded by a term such as “about” or “approximately” include the recited numbers. For example, “approximately 7 mm” includes “7 mm” and numbers and ranges preceded by a term such as “about” or “approximately” should be interpreted as disclosing numbers and ranges with or without such a term in front of the number or value such that this application supports claiming the numbers, values and ranges disclosed in the specification and/or claims with or without the term such as “about” or “approximately” before such numbers, values or ranges such, for example, that “approximately two times to approximately five times” also includes the disclosure of the range of “two times to five times.” The scope of the present disclosure is not intended to be limited by the specific disclosures of preferred arrangements in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A blood reintroduction system, comprising:
a canister having a chamber configured to collect blood;
an inlet configured to fluidically connect the chamber to a first tubing in fluid communication with an aspiration catheter;
a first filter within the chamber, wherein the first filter is configured to separate blood from thrombus;
a second filter downstream of the first filter, wherein the second filter is configured to separate blood from thrombus; and
a second tubing connected to an outlet of the chamber, wherein the second tubing is configured to reintroduce filtered blood from the chamber to a patient's vasculature.

2. The blood reintroduction system of claim 1, wherein a porosity of the first filter is between about 50 microns and about 2000 microns.

3. The blood reintroduction system of claim 1, wherein a porosity of the second filter is between about 20 microns and about 120 microns.

4. The blood reintroduction system of claim 1, wherein the blood reintroduction system is configured to reside within a sterile field.

5. The blood reintroduction system of claim 1, wherein the second filter is positioned outside the chamber and along the second tubing.

6. The blood reintroduction system of claim 1, wherein the second filter is located within the chamber below the first filter.

7. The blood reintroduction system of claim 6, further comprising a third filter positioned outside the chamber and along the second tubing.

8. The blood reintroduction system of claim 1, further comprising a pump configured to decrease pressure within the chamber.

9. The blood reintroduction system of claim 1, further comprising a syringe configured to decrease pressure within the chamber.

10. The blood reintroduction system of claim 1, wherein the second tubing is fluidically connected to a floor of the canister.

11. The blood reintroduction system of claim 10, wherein the floor is positioned on a bottom portion of the canister.

12. The blood reintroduction system of claim 1, wherein the outlet comprises a luer fitting.

13. The blood reintroduction system of claim 1, further comprising an aspiration catheter configured to apply aspiration to the vasculature of the patient.

14. The blood reintroduction system of claim 1, wherein the canister comprises a floor and wherein the floor comprises at least one sloped portion; wherein the at least one sloped portion is configured to direct flow of the blood collected inside the canister towards the outlet.

15. The blood reintroduction system of claim 1, further comprising a third tubing comprising a first end and a second end, the first end configured to be in fluid communication with the second tubing and the second end configured to be in fluid communication with the vasculature of the patient.

16. The blood reintroduction system of claim 15, wherein the third tubing is configured to be in fluid communication with an aspiration source, and the aspiration source is configured to move the blood from the chamber to the vasculature of the patient via the third tubing.

17. The blood reintroduction system of claim 16, further comprising a first one-way valve along the second tubing and a second one-way valve along the third tubing, wherein the first one-way valve is configured to prevent flow from the third tubing to the second tubing.

18. The blood reintroduction system of claim 17, wherein the second one-way valve is configured to prevent flow from a first portion of the third tubing distal to the second one-way valve to a second portion of the third tubing proximal to the second one-way valve.

19. A method of reintroducing blood to a patient's vasculature, the method comprising:
providing a canister to be placed in fluid communication with an aspiration catheter;
applying aspiration via the aspiration catheter to remove blood and thrombus material from the patient's vasculature;
drawing the blood and thrombus material removed by the aspiration catheter into a chamber within the canister;
passing the blood through a first filter and a second filter, wherein the first and second filters are configured to separate blood from thrombus;
collecting the blood in a portion of the chamber positioned above a base of the canister; and
directing the blood via a sloped floor to an outlet fluidically connected to a second tubing, wherein the second tubing is configured to reintroduce filtered blood to the patient's vasculature.

20. (canceled)

21. (canceled)

22. (canceled)

23. (canceled)

24. (canceled)

25. (canceled)

26. (canceled)

27. (canceled)

28. A method of reintroducing blood to vasculature, the method comprising:
providing a canister to be placed in fluid communication with an aspiration catheter;
applying aspiration via the aspiration catheter to remove blood and thrombus material from a patient's vasculature;
collecting the blood and thrombus material removed by the catheter inside a chamber of the canister;
drawing the blood through a first filter, wherein the first filter is configured to separate blood from thrombus;
drawing the blood through a second filter, wherein the second filter is configured to separate blood from thrombus;
collecting the blood in a second portion of the chamber positioned above a base of the canister;
directing the blood via a sloped floor to an outlet fluidically connected to a second tubing; and
reintroducing the filtered blood to a patient's vasculature.

29. (canceled)

30. (canceled)

31. (canceled)

32. (canceled)

33. (canceled)

34. (canceled)

35. (canceled)

36. (canceled)
